(19) United States
(12) Patent Application Publication
(10) Pub. No.: US 2023/0181275 A1
(43) Pub. Date: Jun. 15, 2023

(54) ROBOTIC SURGICAL INSTRUMENTS HAVING ONBOARD GENERATORS

(71) Applicant: Cilag GmbH International, Zug (CH)

(72) Inventors: Mark D. Overmyer, Cincinnati, OH (US); Niko Murrell, Evandale, OH (US); David Perdue, Montgomery, OH (US); Eric N. Johnson, Maineville, OH (US); Benjamin D. Dickerson, Cincinnati, OH (US)

(21) Appl. No.: 17/551,394

(22) Filed: Dec. 15, 2021

(51) Int. Cl. A61B 34/00; A61B 18/12; A61B 34/37; A61B 90/50

(52) U.S. Cl. CPC A61B 34/74; A61B 18/1206; A61B 34/37; A61B 90/50; A61B 34/25

(57) ABSTRACT

Various exemplary devices, systems, and methods for robotic surgical instruments having onboard generators are provided. In general, a surgical instrument is configured to releasably and replaceably couple to a robotic surgical system. The surgical instrument is configured to receive an input from the robotic surgical system that causes the surgical instrument to generate electrical power.

### FIELD

The present disclosure generally relates to robotic surgical instruments having onboard generators.

### BACKGROUND

Minimally invasive surgical (MIS) instruments are often preferred over traditional open surgical devices due to the reduced post-operative recovery time and minimal scarring. Laparoscopic surgery is one type of MIS procedure in which one or more small incisions are formed in the abdomen and a trocar is inserted through the incision to form a pathway that provides access to the abdominal cavity. The trocar is used to introduce various instruments and tools into the abdominal cavity, as well as to provide insufflation to elevate the abdominal wall above the organs. The instruments and tools can be used to engage and/or treat tissue in a number of ways to achieve a diagnostic or therapeutic effect. Endoscopic surgery is another type of MIS procedure in which elongate flexible shafts are introduced into the body through a natural orifice.

Over the years a variety of minimally invasive robotic systems have been developed to increase surgical dexterity as well as to permit a surgeon to operate on a patient in an intuitive manner. Telesurgery is a general term for surgical operations using systems where the surgeon uses some form of remote control, e.g., a servomechanism, or the like, to manipulate surgical instrument movements, rather than directly holding and moving the tools by hand. In such a telesurgery system, the surgeon is typically provided with an image of the surgical site on a visual display at a location remote from the patient. The surgeon can typically perform the surgical procedure at the location remote from the patient whilst viewing the end effector movement on the visual display during the surgical procedure. While viewing typically a three-dimensional image of the surgical site on the visual display, the surgeon performs the surgical procedures on the patient by manipulating master control devices at the remote location, which master control devices control motion of the remotely controlled instruments.

The robotic surgical system provides various inputs to the surgical instrument to control various aspects of the surgical instrument. The surgical instrument is typically mechanically coupled to the robotic surgical system to receive inputs from the robotic surgical system. However, the mechanical coupling only allows for a limited number of inputs, e.g., due to size constraints.

Additionally, surgical instruments used with a robotic surgical system may need electrical power to power various functions of the surgical instrument. However, a surgical instrument may not have a battery or other onboard power source and may not be configured to be plugged into AC power. Onboard power sources and mechanisms for AC power coupling generally make surgical instruments more expensive and heavier by requiring components related to power, which may make the surgical instruments too expensive for some user and/or may make the surgical instruments more difficult to securely connect to and be manipulated a robotic surgical system. Providing electrical power to the surgical instrument from the robotic surgical system is not always practical or efficient because it requires processing resources of the robotic surgical system and requires that limited real estate on the robot side and on the instrument side be dedicated to delivering power from the robotic surgical system to the surgical instrument. Even providing electrical power wirelessly to the surgical instrument from the robotic surgical system (or from another source) can cause real estate problems since wireless antennas are often large and may restrict materials that can be used to make various components of the surgical instrument and/or the robotic surgical system so that a component's material does not interfere with or prevent wireless transmission.

While significant advances have been made in the field of robotic surgery, there remains a need for improved methods, systems, and devices for use in robotic surgery.

### SUMMARY

In general, devices, systems, and methods for robotic surgical instruments having onboard generators are provided.

In one aspect, a surgical system is provided that in one embodiment includes a tool housing of a surgical instrument configured to releasably couple to a tool driver of a robotic surgical system. The tool housing is configured to receive a mechanical, rotational input from the tool driver with the tool housing releasably coupled to the tool driver. The surgical system also includes a generator contained in the tool housing. The receipt of the mechanical, rotational input is configured to cause the generator to generate electrical energy configured to be used onboard the surgical instrument.

The surgical system can vary in any number of ways. For example, the generator can include a motor configured to rotate to generate the electrical energy and can include an energy storage mechanism configured to store the generated electrical energy prior to the use of the generated electrical energy onboard the surgical instrument. The generator can also include a rectifier between the motor and the energy storage mechanism. The energy storage mechanism can include at least one of a capacitor and a battery. The surgical system can include a load contained in the tool housing and configured to be powered with the electrical energy stored in the energy storage mechanism. The load can include a sensing circuit. The load can include an end of life indicator.

For another example, the generated electrical energy can be configured to be used onboard the surgical instrument without storing the generated electrical energy onboard the surgical instrument.

For still another example, the surgical instrument can not be configured to receive electrical energy from the robotic surgical system via a wired connection or a wireless connection.

For yet another example, the input can be from a motor of the tool driver, the input can be configured to cause an input stack of the surgical instrument to rotate, and the rotation of the input stack can be configured to drive the generator to generate the energy.

For still another example, the receipt of the mechanical, rotational input can be configured to cause the generator to generate the electrical energy and to cause the surgical instrument to perform a clinical function.

For another example, the receipt of the mechanical, rotational input can be configured to cause the generator to generate the electrical energy without causing the surgical instrument to perform a clinical function.

For still another example, the surgical system can also include the tool driver.

In another embodiment, a surgical system includes a tool housing of a surgical instrument configured to releasably couple to a tool driver of a robotic surgical system. The tool housing is configured to receive an input from the tool driver with the tool housing releasably coupled to the tool driver, and the input is configured to cause the surgical instrument to perform a clinical function. The surgical system also includes a generator contained in the tool housing. The receipt of the input is configured to cause the generator to generate electrical energy that is configured to be used onboard the surgical instrument.

The surgical system can have any number of variations. For example, the receipt of the input can be configured to cause the generator to generate the electrical energy and to cause the surgical instrument to perform the clinical function.

For another example, the receipt of the input can be configured to cause the generator to generate the electrical energy without causing the surgical instrument to perform the clinical function. The input can be configured to cause movement of a mechanical element within the tool housing, the movement of the mechanical element being within a backlash area can be configured to cause the generator to generate the electrical energy without causing the surgical instrument to perform the clinical function, and the movement of the mechanical element being beyond the backlash area can be configured to cause the generator to generate the electrical energy and to cause the surgical instrument to perform the clinical function.

For yet another example, the input can be a mechanical, rotational input.

For another example, the generator can include a motor configured to rotate to generate the electrical energy and can include an energy storage mechanism configured to store the generated electrical energy prior to the use of the generated electrical energy onboard the surgical instrument. The generator can also include a rectifier between the motor and the energy storage mechanism. The energy storage mechanism can include at least one of a capacitor and a battery. The surgical system can also include a load contained in the tool housing and configured to be powered with the electrical energy stored in the energy storage mechanism. The load can include a sensing circuit. The load can include an end of life indicator.

For yet another example, the generated electrical energy can be configured to be used onboard the surgical instrument without storing the generated electrical energy onboard the surgical instrument.

For still another example, the surgical instrument can not be configured to receive electrical energy from the robotic surgical system via a wired connection or a wireless connection.

For yet another example, the input can be from a motor of the tool driver, the input can be configured to cause an input stack of the surgical instrument to rotate, and the rotation of the input stack can be configured to drive the generator to generate the energy.

For another example, the surgical system can also include the tool driver.

In another aspect, a surgical method is provided that in one embodiment includes receiving, at a tool housing of a surgical instrument releasably coupled to a tool driver of a robotic surgical system, a mechanical input from the tool driver. The receipt of the mechanical, rotational input causes a generator contained in the tool housing to generate electrical energy used onboard the surgical instrument.

The surgical method can vary in any number of ways. For example, the generator can include a motor that rotates to generate the electrical energy and can include an energy storage mechanism that stores the generated electrical energy. The generator can also include a rectifier between the motor and the energy storage mechanism. The energy storage mechanism can include at least one of a capacitor and a battery. The surgical method can also include powering a load contained in the tool housing with the electrical energy stored in the energy storage mechanism. The load can include a sensing circuit. The load can include an end of life indicator.

For another example, the generated electrical energy can be used onboard the surgical instrument without storing the generated electrical energy onboard the surgical instrument.

For yet another example, the input can be from a motor of the tool driver, the input can cause an input stack of the surgical instrument to rotate, and the rotation of the input stack can drive the generator to generate the energy.

For yet another example, the receipt of the mechanical input can cause the generator to generate the electrical energy and can cause the surgical instrument to perform a clinical function.

For still another example, the receipt of the mechanical input can cause the generator to generate the electrical energy without causing the surgical instrument to perform a clinical function.

For another example, the input can be a mechanical, rotational input.

### BRIEF DESCRIPTION OF DRAWINGS

This disclosure will be more fully understood from the following detailed description taken in conjunction with the accompanying drawings, in which:

FIG. 1 is a schematic view of one embodiment of a system including a surgical instrument and a robotic surgical system;

FIG. 2 is a perspective view of one embodiment of a robotic surgical system that includes a patient-side portion and a user-side portion;

FIG. 3 is a perspective view of one embodiment of a robotic arm of a robotic surgical system with a surgical instrument releasably and replaceably coupled to the robotic arm;

FIG. 4 is a side view of the surgical instrument of FIG. 3;

FIG. 5 is a perspective view of a tool driver of the robotic surgical system of FIG. 3;

FIG. 6 is a diagram of one embodiment of a generator;

FIG. 7 is a diagram of another embodiment of a generator;

FIG. 8 is a diagram of yet another embodiment of a generator;

FIG. 9 is a diagram of still another embodiment of a generator;

FIG. 10 is a perspective view of a portion of one embodiment of a tool housing;

FIG. 11 is a diagram of a generator of the tool housing of FIG. 10;

FIG. 12 is a perspective view of a portion of another embodiment of a tool housing;

FIG. 13 is a perspective view of a portion of yet another embodiment of a tool housing with an elongate shaft extending distally therefrom;

FIG. 14 is a perspective view of a portion of the tool housing and the elongate shaft of FIG. 13;

FIG. 15 is a perspective view of another portion of the tool housing of FIG. 13;

FIG. 16 is a perspective view of a portion of another embodiment of a tool housing and an elongate shaft;

FIG. 17 is a perspective view of a portion of another embodiment of a tool housing;

FIG. 18 is a perspective view of another embodiment of a surgical instrument releasably and replaceably coupled to a robotic arm and positioned in an entry guide;

FIG. 19 is a perspective view of a portion of still another embodiment of a tool housing;

FIG. 20 is a perspective view of a portion of yet another embodiment of a tool housing;

FIG. 21 is a graph showing time versus each of input, energy generation, and surgical instrument function;

FIG. 22 is a perspective view of a portion of another embodiment of a tool housing;

FIG. 23 is a perspective view of a portion of yet another embodiment of a tool housing;

FIG. 24 is a diagram of one embodiment of a circuit configured to generate electrical power without storing the power; and

FIG. 25 is a diagram of another embodiment of a circuit configured to generate electrical power without storing the power.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Various exemplary devices, systems, and methods for robotic surgical instruments having onboard generators are provided. In general, a surgical instrument is configured to releasably and replaceably couple to a robotic surgical system. The surgical instrument is configured to receive an input from the robotic surgical system that causes the surgical instrument to generate electrical power. The surgical instrument thus does not need to receive electrical power from the robotic surgical system to power one or more operations of the surgical instrument because the surgical instrument can generate power on its own onboard the surgical instrument.

Examples of robotic surgical systems include the Ottava™ robotic-assisted surgery system (Johnson & Johnson of New Brunswick, N.J.), da Vinci® surgical systems (Intuitive Surgical, Inc. of Sunnyvale, Calif.), the Hugo™ robotic-assisted surgery system (Medtronic PLC of Minneapolis, Minn.), the Versius® surgical robotic system (CMR Surgical Ltd of Cambridge, UK), and the Monarch® platform (Auris Health, Inc. of Redwood City, Calif.). Examples of various robotic surgical systems and using robotic surgical systems are further described in U.S. Pat. Pub. No. 2018/0177556 entitled “Flexible Instrument Insertion Using An Adaptive Force Threshold” filed Dec. 28, 2016, U.S. Pat. Pub. No. 2020/0000530 entitled “Systems And Techniques For Providing Multiple Perspectives During Medical Procedures” filed Apr. 16, 2019, U.S. Pat. Pub. No. 2020/0170720 entitled “Image-Based Branch Detection And Mapping For Navigation” filed Feb. 7, 2020, U.S. Pat. Pub. No. 2020/0188043 entitled “Surgical Robotics System” filed Dec. 9, 2019, U.S. Pat. Pub. No. 2020/0085516 entitled “Systems And Methods For Concomitant Medical Procedures” filed Sep. 3, 2019, U.S. Pat. No. 8,831,782 entitled “Patient-Side Surgeon Interface For A Teleoperated Surgical Instrument” filed Jul. 15, 2013, and Intl. Pat. Pub. No. WO 2014151621 entitled “Hyperdexterous Surgical System” filed Mar. 13, 2014, which are hereby incorporated by reference in their entireties.

Examples of surgical instruments include a surgical dissector, a surgical stapler, a surgical grasper, a clip applier, a smoke evacuator, a surgical energy device (e.g., a mono-polar probe, a bi-polar probe, an ablation probe, an electrosurgical pencil, an ultrasound device, etc.), forceps, a needle driver, scissors, a suction tool, an irrigation tool, and a scope (e.g., an endoscope, an arthroscope, an angioscope, a bronchoscope, a choledochoscope, a colonoscope, a cytoscope, a duodenoscope, an enteroscope, an esophagogastro-duodenoscope (gastroscope), a laryngoscope, a nasopharyngo-neproscope, a sigmoidoscope, a thoracoscope, an ureteroscope, an exoscope, etc.).

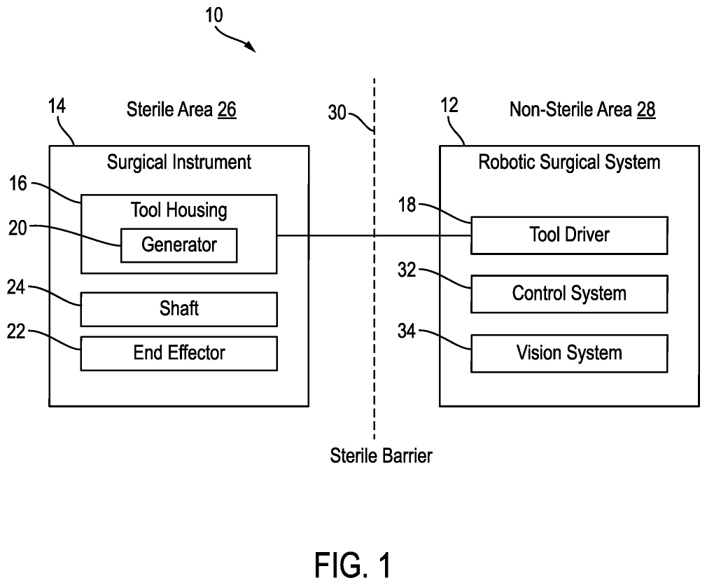

FIG. 1 illustrates one embodiment of a system 10 including a robotic surgical system 12 and a surgical instrument 14. The surgical instrument 14 includes a tool housing (also referred to herein as a “puck”) 16 configured to be releasably and replaceably coupled to a tool driver 18 of the robotic surgical system 12. The surgical instrument 14 is configured to receive an input at the tool housing 16 from the robotic surgical system 12, e.g., from the tool driver 18, that causes a generator 20 onboard the surgical instrument 14 to generate electrical power. The tool housing 16 has the generator 20 contained therein.

In an exemplary embodiment, the input from the robotic surgical system 12 is configured to cause performance of a function of the surgical instrument 14 other than generating electrical power. The surgical instrument 14 can thus be configured to generate electrical power as a side effect of an input received at the surgical instrument 14 for another purpose. The robotic surgical system 12 therefore does not need any modification from its ordinary functioning of providing input to the surgical instrument 14 to allow for electrical power to be generated onboard the surgical instrument 14. In other words, an input that the robotic surgical system 12 is already configured to provide to the surgical instrument 14 for a clinical function can allow for the electrical power generation at the surgical instrument 14. Examples of functions of the surgical instrument 14 that the input can be configured to cause include closing of the surgical instrument's end effector 22 (e.g., closing jaws of the end effector 22), opening of the end effector 22 (e.g., opening jaws of the end effector 22), articulation of the end effector 22 relative to an elongate shaft 24 of the surgical instrument 14 (e.g., angling the end effector 22 relative to a longitudinal axis of the elongate shaft 24), rotation of the end effector 22 relative to the elongate shaft 24 (e.g., rotation of the end effector 22 about a longitudinal axis thereof), rotation of the end effector 22 and the shaft 24 as a unit about the longitudinal axis of the shaft 24, longitudinal movement of the shaft 24 and the end effector 22 along the longitudinal axis of the shaft 24, causing a sensor of the surgical instrument 14 to measure a parameter, ejecting staples from the end effector 22, delivering energy via an electrode of the surgical instrument 14 at the end effector 22, ejecting a clip from the end effector 22, and moving of a cutting element of the surgical instrument 14 along the end effector 22 to cut tissue.

In an exemplary embodiment, the input from the robotic surgical system 12 is a mechanical input to the surgical instrument 14. The surgical instrument 14 can therefore be configured to convert a mechanical input from the robotic surgical system 12 to electrical power, e.g., using the generator 20. The robotic surgical system 12 thus does not need to be configured to deliver electrical power, wired or wirelessly, to the surgical instrument 14 for performing one or more operations of the surgical instrument 14 since the surgical instrument 14 can generate its own electrical power for the performance of the one or more operations. Similarly, the surgical instrument 14 does not need to be configured to receive electrical power, wired or wirelessly, from the robotic surgical system 12.

The robotic surgical system 12 does not need to electrically connect to the surgical instrument 14 at all, wired or wirelessly, since the generator 20 may provide the surgical instrument 14 with needed power. The surgical instrument 14 need not electrically connect at all, wired or wirelessly, to an external power source or have an onboard non-rechargeable battery since the generator 20 may provide the surgical instrument 14 with needed power. The surgical instrument 14 can, however, receive electrical power from the robotic surgical system 12 and/or another external power source, in at least some embodiments, which may allow for more robust powered functions of the surgical instrument 14 than can be provided solely by the onboard generated power.

The power generated local to the surgical instrument 14, e.g., using the generator 20, can be used to power any of a number of operations of the surgical instrument 14. In general, the generated power is configured to power a load (also referred to herein as a “load circuit”).

For example, the power generated by the generator 20 can be used in tracking end of life of the surgical instrument 14. A surgical instrument's end of life can correspond to, for example, a total amount of time the surgical instrument has been in use reaching or exceeding a maximum threshold amount of time or, for another example, a total number of uses of the surgical device reaching or exceeding a maximum threshold number of uses. The surgical instrument's end of life may mean that the surgical instrument needs reconditioning before being used again or may mean that the surgical instrument should be disposed of and not reused. The power generated by the generator 20 can be used to power a load circuit in the form of a life counter or indicator circuit configured to track end of life, such as with a counter (e.g., to count number of instrument uses) or with a timer (e.g., to track a total amount of time the surgical instrument is in use). The life counter or indicator circuit can include a light (e.g., an LED or other type of light) configured to be illuminated when the end of life is reached. The light may therefore be able to be illuminated even without any power being supplied to the surgical instrument 14 from the robotic surgical system 12 (or from any other source) to power the light. Instead of or in addition to the light, end of life may be indicated in another way, such as with a color-changing thermal paste.

For another example, the power generated by the generator 20 can be used to provide power to a load circuit in the form of a sensing circuit of the surgical instrument that is configured to monitor at least one parameter. The sensing circuit may therefore be able to gather data, and in at least some embodiments communicate the data to the robotic surgical system and/or other external system, even without any power being supplied to the surgical instrument 14 from the robotic surgical system 12 (or from another external source or onboard non-rechargeable battery) for the sensor or at all. Examples of parameters include pressure, temperature, impedance, and motion. The sensing circuit includes at least one sensor configured to monitor the at least one parameter. Examples of sensors include switches, buttons, thermometers, Hall effect sensors, and strain gauges. The sensing circuit can be configured to communicate wirelessly, such as by using Bluetooth, Wifi, radio frequency identification (RFID), or optical communication.

For yet another example, the power generated by the generator 20 can be used to provide power to a load circuit in the form of a microchip onboard the surgical instrument 14 that is configured to store operational parameters related to the surgical instrument 14, such as in a storage mechanism of the microchip, and in at least some embodiments communicate the data to the robotic surgical system and/or other external system. Operational parameters may therefore be able to be updated or to be stored for the first time even without any power being supplied to the surgical instrument 14 from the robotic surgical system 12 (or from another external source or onboard non-rechargeable battery) for managing operational parameters or at all. Examples of storage mechanisms include non-volatile microcontroller memory, read-only memory (ROM) (e.g., erasable programmable ROM (EPROM) and electronically erasable programmable ROM (EEPROM)), flash memory, and random access memory (RAM) (e.g., static RAM (SRAM), dynamic RAM (DRAM), or synchronous DRAM (SDRAM)). Examples of operational parameters include end effector 22 opening speed, end effector 22 closing speed, cutting element speed, level of energy application, motor speed, time, light emission, staple size, measurements made during manufacturing of the surgical instrument (or particular components thereof), previous instrument use statistics, data and revision of manufacturing of the surgical instrument (or particular components thereof), and last known status of the surgical instrument.

As shown in FIG. 1, the system 10 includes a sterile area 26 and a non-sterile area 28 that are separated from one another by a sterile barrier 30. The sterile barrier 30 is configured to provide a sterile operation area. The sterile area 26 is an area including a patient on which a surgical procedure is being performed. The sterile area 26 is on a sterile side of the sterile barrier 30, and the non-sterile area 28 is on a non-sterile side of the sterile barrier 30. The surgical instrument 14 is located in the sterile area 26. The non-sterile area 28 is an area located a distance from the patient, either in the same room and/or in a remote location. The robotic surgical system 12 is located in the non-sterile area 28. A user can thus visualize and control the surgical instrument 14, which is in a sterile environment, from a non-sterile environment. The sterile barrier 30 can have a variety of configurations. For example, the sterile barrier 30 can include a sterile drape. Various other examples of sterile barriers are described further in U.S. Pat. No. 10,433,920 entitled “Surgical Tool And Robotic Surgical System Interfaces” issued Oct. 8, 2019 and U.S. Pat. No. 10,433,925 entitled “Sterile Barrier For Robotic Surgical System” issued Oct. 8, 2019, which are hereby incorporated by reference in their entireties.

The robotic surgical system 12 includes a control system 32 configured to allow a user to control the surgical instrument 14 releasably and replaceably coupled to the robotic surgical system 12. The control system 32 can have a variety of configurations and can be located adjacent to a patient (e.g., in the operating room), can be located remote from the patient (e.g., in a separate control room), or can be distributed at two or more locations. For example, a dedicated system control console can be located in the operating room, and a separate console can be located in a remote location. The control system 32 can include one or more manually-operated input devices, such as a joystick, exoskeletal glove, a powered and gravity-compensated manipulator, or the like. In general, the input device is configured to control teleoperated motors which, in turn, control elements including the surgical instrument 14.

The robotic surgical system 12 also includes a vision system 34 configured to allow the user to visualize the surgical instrument 14 and/or surgical site. The vision system 34 can have a variety of configurations and can be located adjacent to a patient, can be located remote from the patient, or can be distributed at two or more locations.

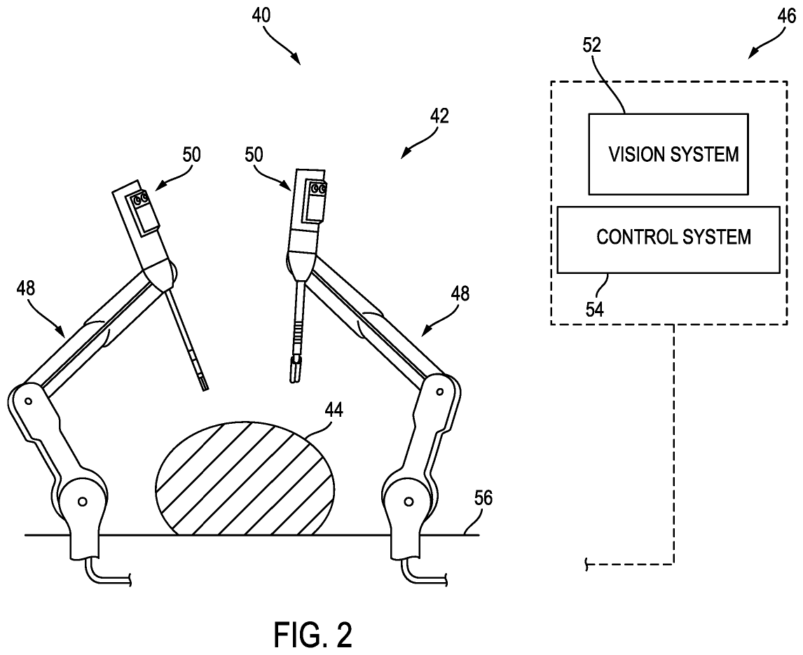

FIG. 2 illustrates one embodiment of a robotic surgical system 40 that can be used as the robotic surgical system 12. The robotic surgical system 40 includes a patient-side portion 42 that is positioned adjacent to a patient 44, and a user-side portion 46 that is located a distance from the patient 44, either in the same room and/or in a remote location. The user-side portion 46 includes a vision system 52 (e.g., the vision system 34) and a control system 54 (e.g., the control system 32). The control system 54 includes an input device is configured to control teleoperated motors which, in turn, control elements including robotic arms 48 and surgical instruments 50.

The patient-side portion 42 includes one or more robotic arms 48 that are each configured to releasably and replaceably coupled to a surgical instrument 50, e.g., the surgical instrument 14 of FIG. 1. As shown in FIG. 2, the patient-side portion 42 can couple to an operating table 56. However, in some embodiments, the patient-side portion 42 can be mounted to a wall, to the ceiling, to the floor, or to other operating room equipment. Further, while the patient-side portion 42 is shown as including two robotic arms 48, more or fewer robotic arms 48 may be included. Furthermore, the patient-side portion 42 can include separate robotic arms 48 mounted in various positions, such as relative to the operating table 56, as shown in FIG. 2. Alternatively, the patient-side portion 42 can include a single assembly that includes one or more robotic arms 48 extending therefrom.

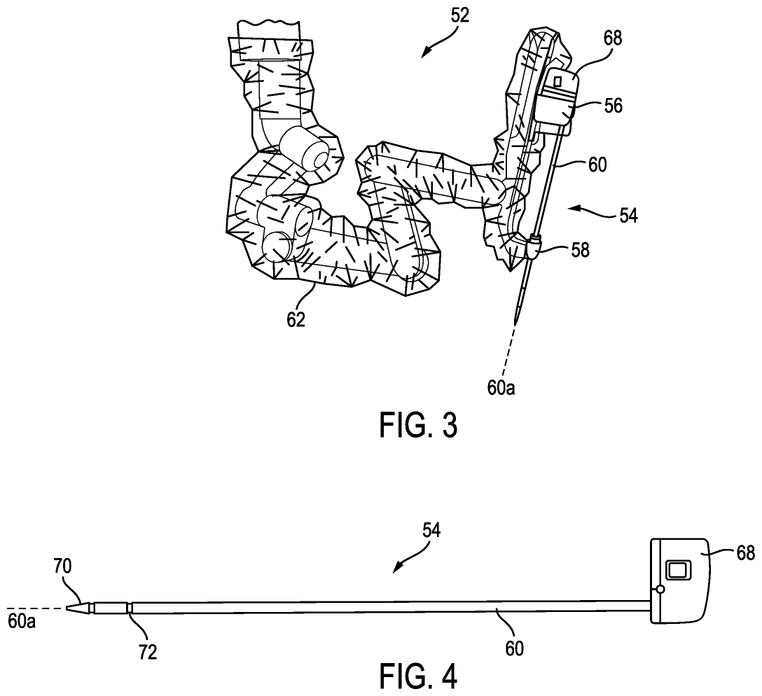

FIG. 3 illustrates one embodiment of a robotic arm 52, which can be used as the robotic arm 48, and a surgical instrument 54, which can be used as the surgical instrument 50, releasably coupled to the robotic arm 52. The surgical instrument 54 is also illustrated in FIG. 4. The robotic arm 52 is configured to support and move the associated surgical instrument 54 along one or more mechanical degrees of freedom (e.g., all six Cartesian degrees of freedom, five or fewer Cartesian degrees of freedom, etc.).

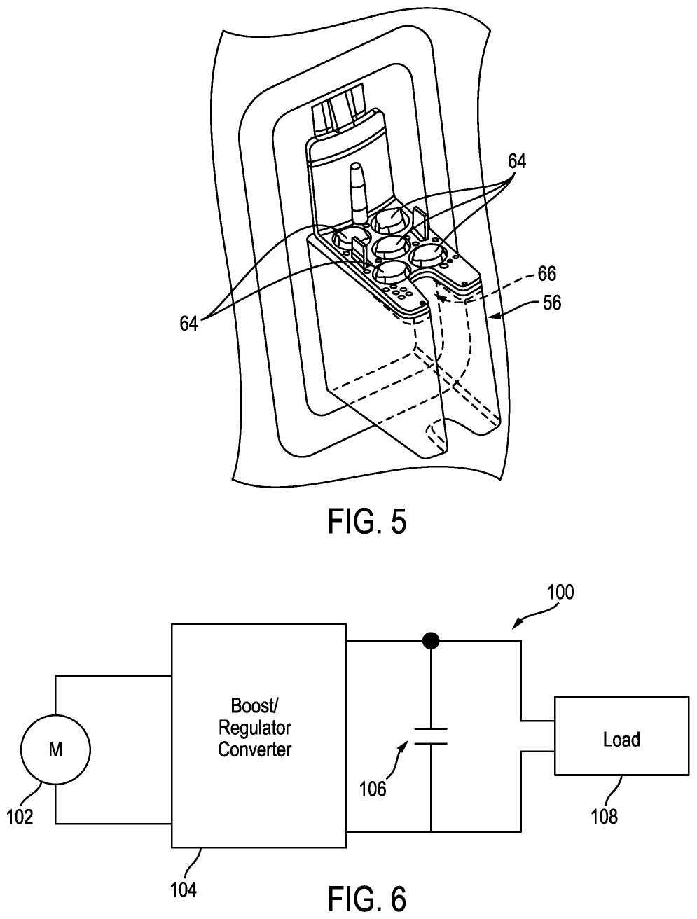

The robotic arm 52 includes a tool driver 56, e.g., the tool driver 18 of FIG. 1, at a distal end of the robotic arm 52. The tool driver 56 is also shown in FIG. 5. The robotic arm 52 also includes an entry guide 58 (e.g., a cannula mount or cannula) that can be a part of or removably coupled to the robotic arm 52, as shown in FIG. 3. An elongate shaft 60 of the surgical instrument 54, e.g., the elongate shaft 24 and end effector 22 of the surgical instrument 14 of FIG. 1, are configured to be inserted through the entry guide 58 for insertion into a patient.

In order to provide a sterile operation area while using the surgical system, the system includes a sterile barrier 62 (e.g., the sterile barrier 30) located between an actuating portion of the system (e.g., the robotic arm 52) and the surgical instruments (e.g., the surgical instrument 54). A sterile component, such as an instrument sterile adapter (ISA), can also be placed at the connecting interface between the surgical instrument 54 and the robotic arm 52. An ISA between the surgical instrument 54 and the robotic arm 52 is configured to provide a sterile coupling point for the surgical instrument 54 and the robotic arm 52. This permits removal of the surgical instrument 54 from the robotic arm 52 for replacement with another surgical instrument during the course of a surgical procedure without compromising the sterile surgical field.

As shown in FIG. 5, the tool driver 56 includes a plurality of motors 64 configured to control a variety of movements and actions associated with the surgical instrument 54. Five motors 64 are shown in this illustrated embodiment, but another plural number of motors may be used or only one motor may be used. Each motor 64 is configured to couple to and/or interact with an activation feature (e.g., gear and/or other elements) of the surgical instrument 54 at a tool housing 68, e.g., the tool housing 16, of the surgical instrument 54. The motors 64 are accessible on an upper surface of the tool driver 56, and thus the surgical instrument 54 is configured to mount on top of the tool driver 56 to couple thereto via the tool housing 68. The tool driver 56 also includes a shaft-receiving channel 66 formed in a sidewall thereof for receiving the elongate shaft 60 of the surgical instrument 54. In other embodiments, the shaft 60 can extend through an opening in the tool driver 56, or the two components can mate in various other configurations.

As shown in FIG. 4, the puck 68 of the surgical instrument 54 is coupled to a proximal end of the shaft 60, and an end effector 70, e.g., the end effector 22, is coupled to a distal end of the shaft 60. As discussed further below, the puck 68 includes one or more coupling element configured to facilitate releasably coupling the puck 68 to the tool driver 56 and thus, in at least some embodiments, to facilitate the surgical instrument's receipt of input from the robotic surgical system to cause a generator, e.g., the generator 20, of the surgical instrument 54 to generate electrical power.

The puck 68 includes gears and/or actuators that can be actuated by the one or more motors 64 of the tool driver 56. The gears and/or actuators in the puck 68 are configured to control various functions of the surgical instrument 54, such as various functions associated with the end effector 70 (e.g., end effector 70 opening, end effector 70 closing, longitudinal movement of the shaft 60 and the end effector 70, staple firing, rotation of the end effector 70 and/or the shaft 60, articulation of the end effector 70, energy delivery, etc.), as well as control the movement of the shaft 60 such as longitudinal translation of the shaft 60 with the end effector 70 and such as rotation of the shaft 60 relative to the puck 68. Various embodiments of pucks and gears and actuators of a puck configured to control various functions of a surgical instrument are described further in, for example, U.S. Pat. Pub. No. 2018/0049820 entitled “Control Of Robotic Arm Motion Based On Sensed Load On Cutting Tool” published Feb. 22, 2018, U.S. Pat. No. 10,231,775 entitled “Robotic Surgical System With Tool Lift Control” issued Mar. 19, 2019, U.S. Pat. No. 10,813,703 entitled “Robotic Surgical System With Energy Application Controls” issued Oct. 27, 2020, U.S. Pat. Pub. No. 2018/0049818 entitled “Control Of The Rate Of Actuation Of Tool Mechanism Based On Inherent Parameters” published Feb. 22, 2018, U.S. Pat. No. 2018/0049795 entitled “Modular Robotic Surgical Tool” published Feb. 22, 2018, U.S. Pat. No. 10,548,673 entitled “Surgical Tool With A Display” issued Feb. 4, 2020, U.S. Pat. No. 10,849,698 entitled “Robotic Tool Bailouts” issued Dec. 1, 2020, U.S. Pat. No. 10,433,925 entitled “Sterile Barrier For Robotic Surgical System” issued Oct. 8, 2019, U.S. Pat. No. 10,675,103 entitled “Robotics Communication And Control” issued Jun. 9, 2020, U.S. Pat. No. 9,943,377 entitled “Methods, Systems, And Devices For Causing End Effector Motion With A Robotic Surgical System” issued Apr. 17, 2018, U.S. Pat. No. 10,016,246 entitled “Methods, Systems, And Devices For Controlling A Motor Of A Robotic Surgical System” issued Jul. 10, 2018, U.S. Pat. No. 10,045,827 entitled “Methods, Systems, And Devices For Limiting Torque In Robotic Surgical Tools” issued Aug. 14, 2018, U.S. Pat. No. 10,478,256 entitled “Robotic Tool Bailouts” issued Nov. 19, 2019, U.S. Pat. Pub. No. 2018/0049824 entitled “Robotics Tool Exchange” published Feb. 22, 2018, U.S. Pat. No. 10,398,517 entitled “Surgical Tool Positioning Based On Sensed Parameters” issued Sep. 3, 2019, U.S. Pat. No. 10,363,035 entitled “Stapler Tool With Rotary Drive Lockout” issued Jul. 30, 2019, U.S. Pat. No. 10,413,373 entitled “Robotic Visualization And Collision Avoidance” issued Sep. 17, 2019, and U.S. Pat. No. 10,736,702 entitled “Activating And Rotating Surgical End Effectors” issued Aug. 11, 2020, which are hereby incorporated by reference in their entireties.

The shaft 60 can be fixed to the puck 68, or the shaft 60 can be releasably and replaceably coupled to the puck 68 such that the shaft 60 can be interchangeable with other elongate shafts. This can allow a single puck 68 to be used with different elongate shafts having different configurations and/or different end effectors. The elongate shaft 60 includes various actuators and connectors that extend along the shaft 60 within an inner lumen thereof that are configured to assist with controlling the actuation and/or movement of the end effector 70 and/or shaft 60. As in this illustrated embodiment, the surgical instrument 54 can include at least one articulation joint 72 configured to allow the end effector 70, either alone or with a distal portion of the shaft 60, to articulate relative to a longitudinal axis 60a of the shaft 60. The articulation can allow for fine movements and various angulation of the end effector 70 relative to the longitudinal axis 60a of the shaft 60.

FIG. 6 illustrates one embodiment of generator 100 configured to be housing by a surgical instrument's tool housing, e.g., the tool housing 16 of FIG. 1 or the tool housing 68 of FIGS. 3 and 4, and configured to cause electrical power to be generated and stored at the surgical instrument. The generator 100 includes a DC motor 102 (such as a rotary permanent magnet DC motor), a voltage booster and regulator circuit 104, and an energy storage mechanism 106. The energy storage mechanism 106 includes a capacitor in this illustrated embodiment. A load 108 is configured to be powered by the energy stored by the energy storage mechanism 106. Activation of the motor 102 is configured to cause electrical energy to be stored in the energy storage mechanism 106, through the voltage booster and regulator circuit 104. The voltage booster and regulator circuit 104 is configured to, such as with a bridge rectifier with four diodes, maintain a constant DC voltage with the DC voltage provided by the motor 102 being below or above the voltage needed by the load 108. The load 108 can have a variety of configurations, as discussed herein.

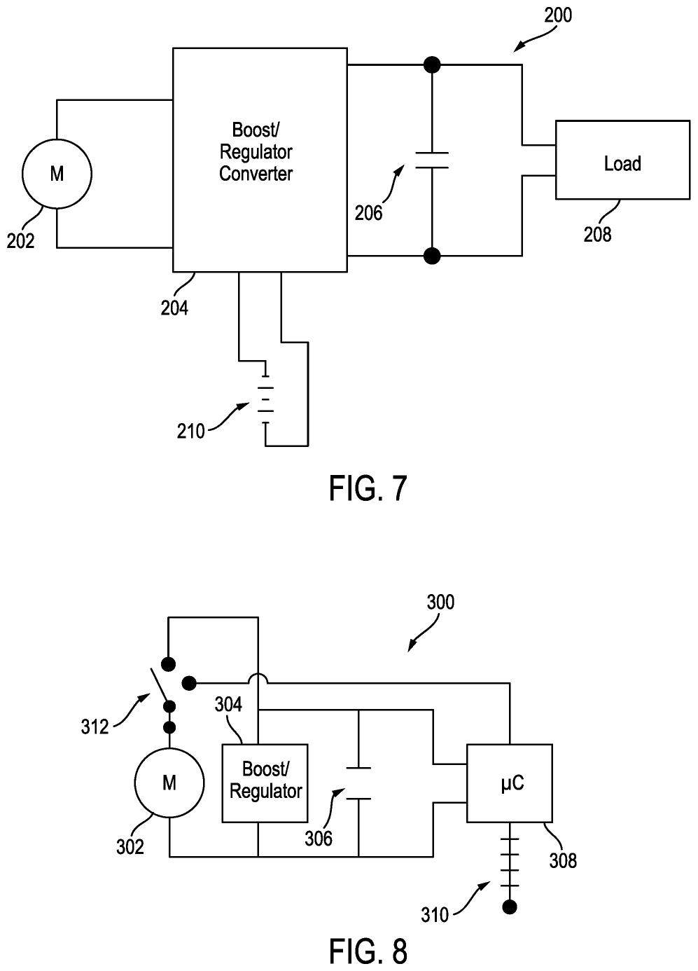

FIG. 7 illustrates another embodiment of generator 200 configured to be housing by a surgical instrument's tool housing, e.g., the tool housing 16 of FIG. 1 or the tool housing 68 of FIGS. 3 and 4, and configured to cause electrical power to be generated and stored at the surgical instrument. The generator 200 includes a DC motor 202 (such as a rotary permanent magnet DC motor), a voltage booster and regulator circuit 204, and an energy storage mechanism. The embodiment of FIG. 7 is similar to the embodiment of FIG. 6 except that the energy storage mechanism of the FIG. 7 embodiment includes a capacitor 206 and a battery 210. Electrical power in this illustrated embodiment is stored in the capacitor 206 and transferred therefrom to the battery 210 at a controlled rate to avoid damaging the battery 210. A load 208 is configured to be powered by the energy stored by the energy storage mechanism, e.g., by each of the capacitor 206 and the battery 210.

FIG. 8 illustrates another embodiment of generator 300 configured to be housing by a surgical instrument's tool housing, e.g., the tool housing 16 of FIG. 1 or the tool housing 68 of FIGS. 3 and 4, and configured to cause electrical power to be generated and stored at the surgical instrument. The generator 300 includes a DC motor 302 (such as a rotary permanent magnet DC motor), a voltage booster and regulator circuit 304, and an energy storage mechanism 306. The energy storage mechanism 306 includes a capacitor in this illustrated embodiment. A load is configured to be powered by the energy stored by the energy storage mechanism 306 and includes a microcontroller 308 and a wireless communication mechanism 310 (e.g., an antenna or other mechanism) in this illustrated embodiment. The embodiment of FIG. 8 is similar to the embodiment of FIG. 6 except that a robotic surgical system operatively coupled to the surgical instrument is configured to selectively enable and disable the generator 300. The generator 300 includes a switch 312 configured to be selectively opened and closed to enable (switch 312 closed) and disable (switch 312 open) power generation by the generator 300. The switch 312 is open in FIG. 8. With the switch 312 open, the generator 300 cannot generate current and is disconnected from the load circuit. With the switch 312 closed, the generator 300 can generate current and is connected to the load circuit. The switch 312 is an electrically activated switch operatively coupled to the microcontroller 308. The robotic surgical system is configured to transmit an instruction signal to the microcontroller 308 via the communication mechanism 310. In response to receiving the instruction signal, the microcontroller 308 is configured to cause the switch 312 to move to either open the switch 312 or close the switch 312 depending on the switch's current state of open or closed.

It may be advantageous for the robotic surgical system to disable the generator 300 when mechanical power being provided to the surgical instrument from the robotic surgical system (e.g., to the tool housing from the tool driver) is high such that generating electrical power using the generator 300 in addition to performing other function(s) per the robotic surgical system's input(s) may risk exceeding abilities of the tool housing's gears and/or actuators. For example, an input for transecting tissue generally requires high mechanical power on the instrument side. The robotic surgical system can thus be configured to transmit an instruction signal to the microcontroller 308 via the communication mechanism 310 when the robotic surgical system provides an input to an input stack of the surgical instrument's tool housing to transect tissue. The instruction signal can be provided simultaneously with the input or in near real time therewith. The input stack to which the input is provided therefore does not need to use any mechanical power for the generator 300, instead using its mechanical power for transecting tissue. When the tissue transection is complete, the robotic surgical system can send a second instruction signal to the microcontroller 308 via the communication mechanism 310 to close the switch 312 to allow for energy generation.

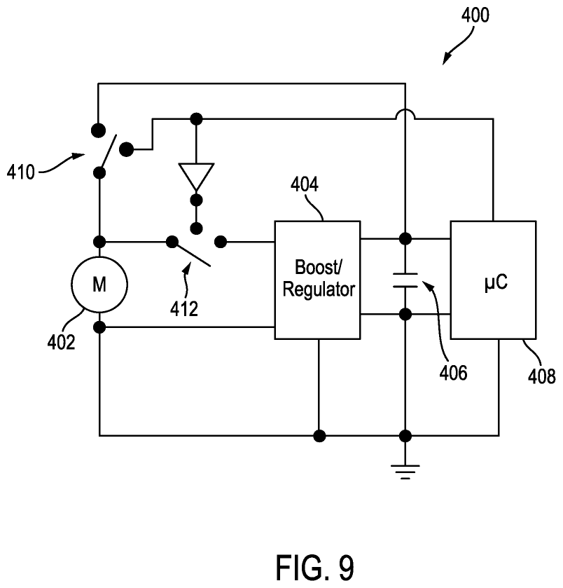

FIG. 9 illustrates another embodiment of generator 400 configured to be housing by a surgical instrument's tool housing, e.g., the tool housing 16 of FIG. 1 or the tool housing 68 of FIGS. 3 and 4, and configured to cause electrical power to be generated and stored at the surgical instrument. The generator 400 includes a DC motor 402 (such as a rotary permanent magnet DC motor), a voltage booster and regulator circuit 404, and an energy storage mechanism 406. The energy storage mechanism 406 includes a capacitor in this illustrated embodiment. A load circuit is configured to be powered by the energy stored by the energy storage mechanism 406 and includes a microcontroller 408 in this illustrated embodiment. The embodiment of FIG. 9 is similar to the embodiment of FIG. 8 except that the microcontroller 408 is configured to cause a first switch 410 to selectively open and close to enable (first switch 410 closed) and disable (first switch 410 open) power generation by the generator 300, and is configured to cause a second switch 412 to selectively open and close to disconnect (second switch 412 open) and connect (second switch 412 closed) the motor 402 and the energy storage mechanism 406. The first and second switches 410, 412 are each open in FIG. 9.

The first and second switches 410, 412 are configured to allow the load circuit to function after the surgical instrument is released from a robotic surgical system. The load circuit functioning after such release may facilitate recoupling of the tool housing with a robotic surgical system after being released from the robotic surgical system (or from another robotic surgical system). For example, the microcontroller 408 can be configured to sense release of the surgical instrument from a robotic surgical system, e.g., the tool housing decoupled from the robotic surgical system's tool driver. In response to sensing the release of the surgical instrument from the robotic surgical system, the microcontroller 408 can be configured to cause the second switch 412 to open, thereby disconnecting the motor 402 from the energy storage mechanism 406 and the voltage booster and regulator circuit 404. The microcontroller 408 can then feed energy stored in the energy storage mechanism 406 to the motor 402, which the first switch 410 being closed allows. The motor 402, receiving power, can thus drive a mechanical element of the tool housing which drives the motor 402 to cause power generation. The mechanical element can thus be configured to be in a mechanical state configured for recoupling to the robotic surgical system (or another robotic surgical system). The mechanical element can include, for example, a transection gear train with the motor 402 driving the transection gear train to retract proximally until the transaction gear train hits a hard stop to position the transection gear train at a start position for a next transection.

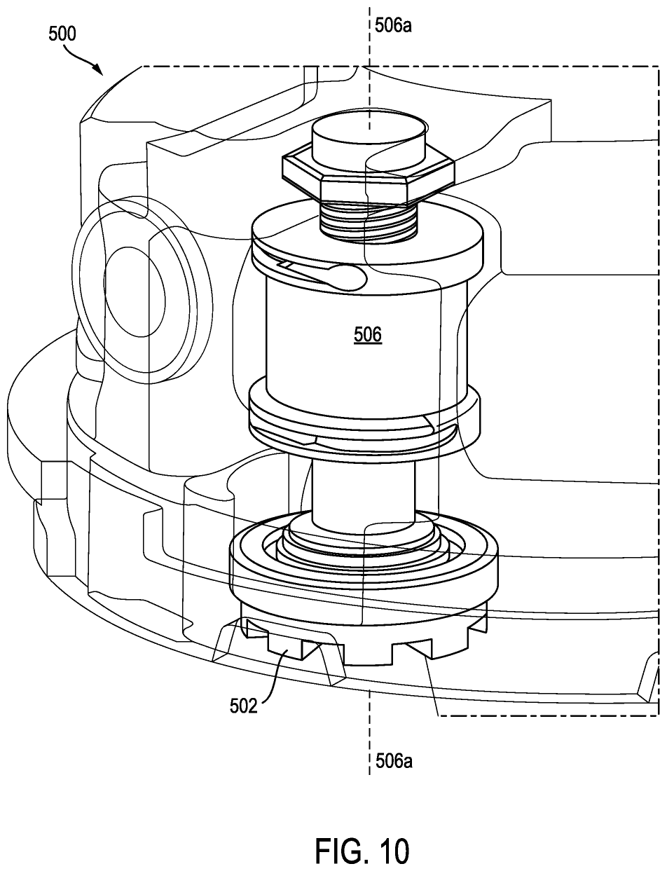

FIG. 10 illustrates another embodiment of a tool housing 500, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing 500 is only partially shown in FIG. 10. As discussed further below, an input from the tool driver to the tool housing 500 is configured to cause electrical power to be generated and stored at the surgical instrument using a generator housed in the tool housing 500.

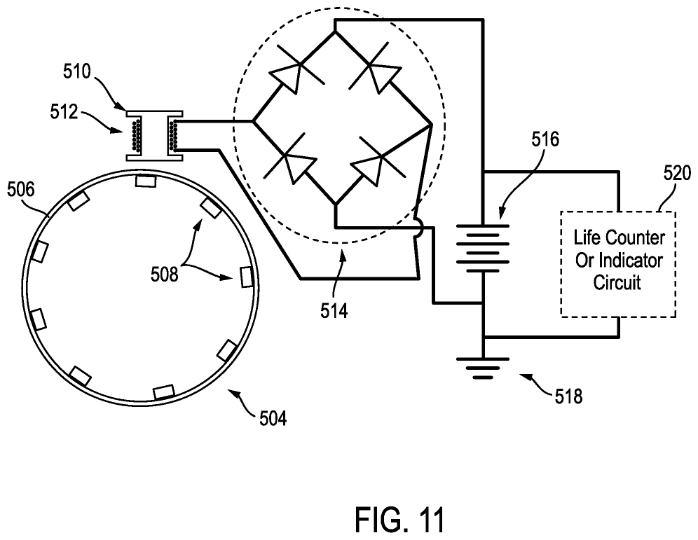

The tool housing 500 includes a coupling element 502 configured to operatively couple to a motor of a tool driver (e.g., one of the motors 64 of the tool driver 56). The coupling element 502 in this illustrated embodiment includes a gear with teeth configured to operatively engage corresponding teeth of the motor. The coupling element 502 is part of an insertion input stack 504 (also see FIG. 11) of gears and actuators configured to be actuated to cause longitudinal movement of an elongate shaft and an end effector of the surgical instrument along the shaft's longitudinal axis (e.g., longitudinally move the shaft 60 and the end effector 70 along the longitudinal axis 60a). The longitudinal movement can be distal advancement or proximal retraction depending on how a user desires to position the surgical instrument.

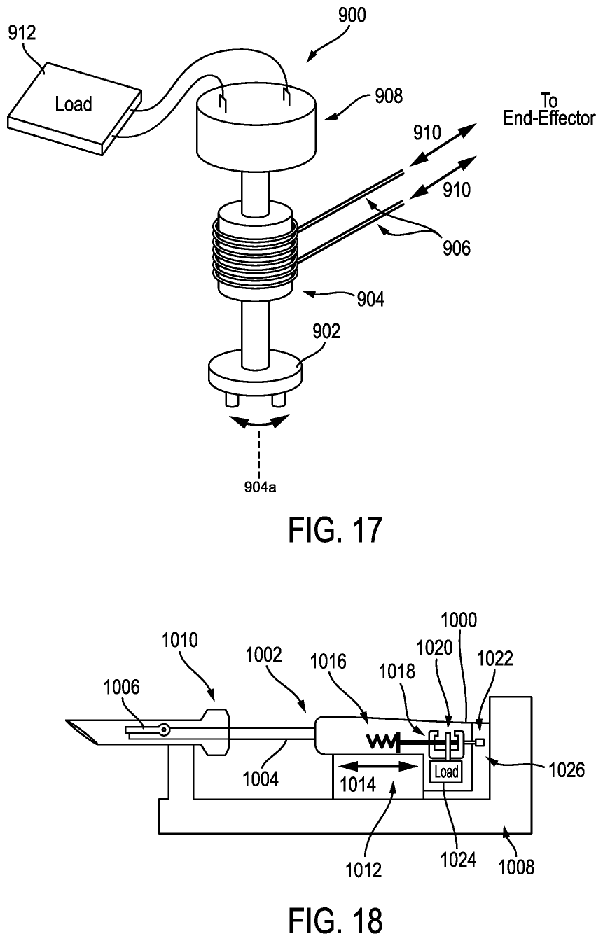

The insertion input stack 504 also includes a drum 506. The drum 506 is configured to rotate about a longitudinal axis 506a of the drum 506 that also defines a longitudinal axis of the insertion stack. The drum's longitudinal axis 506a is substantially parallel to a longitudinal axis of the surgical instrument's elongate shaft. A person skilled in the art will appreciate that axes may not be precisely parallel but nevertheless considered to be substantially parallel for any of a variety of reasons, such as sensitivity of measurement equipment and manufacturing tolerances. As will also be appreciated by a person skilled in the art, the drum 506 is configured to operatively couple to a wire or cable (see, e.g., FIG. 17) that is operatively coupled to the elongate shaft. In this way, rotation of the drum 506 can cause movement of the wire or cable and thus cause longitudinal movement of the elongate shaft and the end effector coupled thereto.

The tool driver coupled to the tool housing 500 via the coupling element 502 is configured to provide an input to the tool housing 500 that causes the coupling element 502 to rotate and thus cause the drum 506 to rotate, thereby causing longitudinal translation of the elongate shaft and the end effector. The input thus includes a rotational input and includes the motor being driven to provide a rotational, mechanical input to the tool housing 500, e.g., the toothed gear of the motor rotating to cause corresponding rotation of the coupling element 502.

As shown in FIG. 11, the insertion input stack 504 is operatively coupled to a generator configured to generate electrical power. The generator is omitted in FIG. 10 for clarity of illustration. The generator is contained within the tool housing 500. In general, the rotation of the insertion input stack is configured to cause the generator to generate electrical power. Thus, the mechanical input to the tool housing 500 from the tool driver can cause the surgical instrument to generate electrical power onboard.

Although the generator of FIG. 11 is described with respect to the insertion input stack 504 configured to be actuated to cause longitudinal translation of the surgical instrument's elongate shaft and end effector for insertion and retraction, the generator can be similarly used with other input stacks in the tool housing 500 that are each configured to be actuated by one or more motors of the tool driver, such as an articulation input stack configured to receive an input from the tool driver to cause articulation of the end effector, a rotation input stack configured to receive an input from the tool driver to cause rotation of the end effector and the elongate shaft relative to the tool housing 500, an end effector movement stack configured to receive an input from the tool driver to cause opening and/or closing of the end effector, a firing input stack configured to receive an input from the tool driver to cause staple firing from the end effector, etc. The input received by each of the various input stacks is similar to that discussed above regarding the insertion input stack 504, e.g., a rotational, mechanical input from the tool driver. The generator can also similarly be used with insertion input stacks having a different configuration than the illustrated insertion input stack 504.

The generator being operatively coupled to an insertion input stack such as the insertion input stack 504 or other configuration of an insertion input stack may most efficiently generate power onboard the surgical instrument as compared to other input stacks. The insertion input stack 504 (or other configuration of an insertion input stack) rotates faster than other input stacks due to a higher input speed from the tool driver to cause elongate shaft and end effector translation as compared to input speeds needed to effectively actuate other input stacks. The insertion input stack 504 (or other configuration of an insertion input stack) may be the first of all a tool housing's input stack to be activated by a robotic surgical system so the surgical instrument's elongate shaft and end effector can be desirably positioned before other actions are taken with the surgical instrument, so operatively coupling the generator to the insertion input stack 504 may allow for electrical energy to be generated early in the use of the surgical instrument.

The generator includes a magnet 508, a ferromagnetic core 510, a copper wire 512 winding around the ferromagnetic core 510, a rectifier 514, and an energy storage mechanism 516. The magnet 508 in this illustrated embodiment includes nine magnets, but another plural number of magnets can be used or only one magnet can be used. The ferromagnetic core 510 is made from iron in this illustrated embodiment but other ferromagnetic materials can be used, e.g., nickel, cobalt, etc. The generator includes only one ferromagnetic core 510 and associated copper coil 512 in this illustrated embodiment but can include a plurality of ferromagnetic cores each with an associated copper coil. The energy storage mechanism 516 is configured to store the generated power. The energy storage mechanism 516 can have a variety of configurations, such as a battery or a capacitor. The power generation performed by the generator in this illustrated embodiment is non-contact electromagnetic, which will not add frictional resistance to the insertion stack axis (which is coaxial with the drum's longitudinal axis 504a).

The plurality of magnets 508 are arranged around a circumference of the drum 504. The magnets 508 are located internal to the drum 504, which may help reduce an overall footprint of the drum 504 within the tool housing 500. Because the magnets 508 are attached to the drum 504, the rotation of the drum 504 causes the magnets 508 to rotate. The rotation of the magnets 508 causes the magnets 508 to interact with the copper coil 512 and generate an electromagnetic field. The rectifier (also referred to herein as a “generator circuit”) 514 is configured to convert the AC electromagnetic field to DC current, which is output to the energy storage mechanism 516 for storage therein. The generator circuit 514, which is simplified as shown in FIG. 7, includes at least one diode, silicon controlled rectifier (SCR) circuit, or other semiconductor component configured to rectify the electrical current produced by the cooperation of the magnets 510 and the copper wire 512 to be suitable for charging the energy storage mechanism 516.

The energy storage mechanism 516 is coupled to ground 518 and to load in the form of a life counter or indicator circuit 520. The power generated by the generator is configured to power the life counter or indicator circuit 520. The life counter or indicator circuit 520 is configured to track use of the surgical instrument for end of life purposes, as discussed above. As also discussed above, the life counter or indicator circuit 520 can include a light (e.g., an LED or other type of light) configured to be illuminated when the end of life is reached in addition to or instead of another end of life indicator. The energy storage mechanism 516 is configured to power the life counter or indicator circuit 520 in this illustrated embodiment but can be similarly used to power another load.

In the embodiment of FIGS. 10 and 11, the input from the tool driver to the tool housing 500 is configured to cause the generator to generate power and cause an output function of the surgical instrument, which in this illustrated embodiment is elongate shaft and end effector translation. The generator can thus generate power onboard the surgical instrument during the surgical instrument's ordinary use of performing a clinical function.

In other embodiments, an input from a tool driver to a tool housing is configured to cause a generator to generate power without causing an output function of the surgical instrument. The generator can thus generate power while the surgical instrument is idle without any of the tool driver's motors driving any function of the surgical instrument, or while the surgical instrument is performing another function in response to another input. Such a configuration takes advantage of a tool housing being configured to be driven by each of a plurality of motors of the tool driver because at least one of the motors can be causing the electrical energy to be generated onboard the surgical instrument by actuating a first input stack of the tool housing while the surgical instrument is otherwise idle (no other motors are driving a function of the surgical instrument) or performing another function (at least one of the other motors is driving a function of the surgical instrument by actuating at least one other of the tool housing's input stacks).

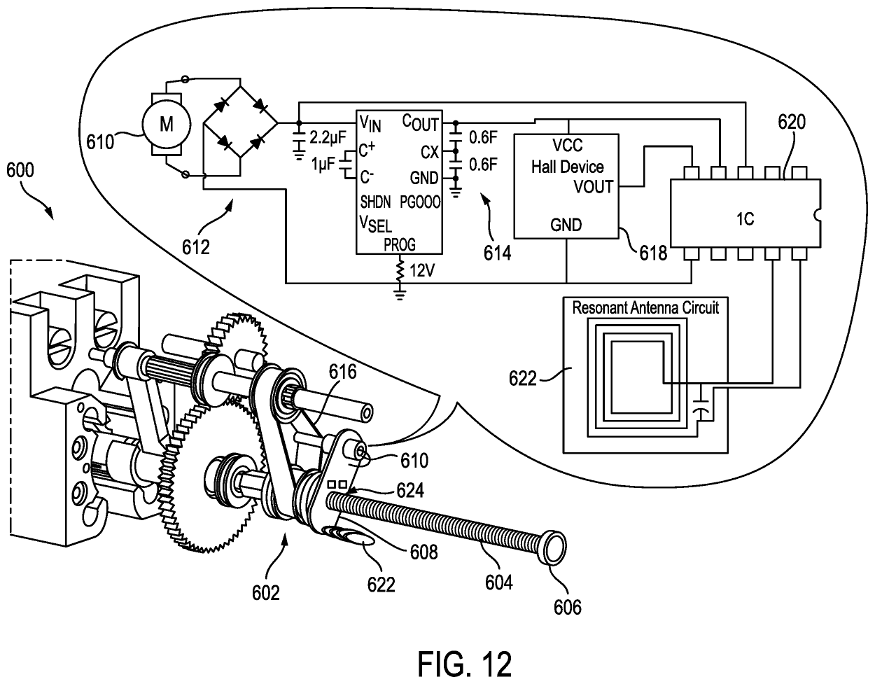

FIG. 12 illustrates another embodiment of a tool housing 600, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing 600 is only partially shown in FIG. 12. The tool housing 600 includes a generator configured to cause electrical power to be generated at the surgical instrument and that is. A tool driver to which the tool housing 600 is coupled is configured to cause the generator to generate and store power without causing an output function of the surgical instrument. The embodiment of FIG. 12 can use backlash to generate electrical energy at the tool housing 600. In this illustrated embodiment, the backlash is rotational backlash.

The tool housing 600 is configured to operatively couple to one or more motors of the tool driver via one or more input stacks of the tool housing, similar to that discussed above. In this illustrated embodiment, a firing input stack 602 is configured to receive an input from the tool driver to cause staple firing from the surgical instrument's end effector. The firing input stack 602 is also configured to receive an input from the tool driver to cause the generator to generate electrical power without causing any staple firing from the end effector. The firing input stack 602 is not activated while any other input stack of the tool housing 600 is activated, e.g., firing does not occur during functions of the surgical instrument such as end effector articulation, end effector and shaft translation, or end effector opening or closing. The firing input stack 602 can therefore be used for generating electrical power without interfering with other functions of the surgical instrument or mechanically overloading the tool housing 600.

The firing input stack 602 is operatively coupled to a leadscrew drivetrain 604 that is configured to rotate to drive firing of staples from the end effector. The generator includes a magnet 606 that is attached to the leadscrew drivetrain 604. A circuit board 608 is also attached to the leadscrew drivetrain 604. The circuit board 608 is located a distance away from the magnet 606 proximal to the magnet 606. The inset of FIG. 12 illustrates various elements on the circuit board 608. The generator includes a DC motor 610 (such as a rotary permanent magnet DC motor), a rectifier 612 on the circuit board 608, and an energy storage mechanism 614 on the circuit board 608. The circuit board 608 includes thereon a load circuit in the form of a sensing circuit including a Hall effect sensor 618, a microchip (IC circuit) 620, and a resonant antenna circuit 622. The motor 610 is operatively coupled to a belt 616, which is also operatively coupled to the leadscrew drivetrain 604.

Input to the firing input stack 602 from the tool driver operably coupled to the tool housing 600 is configured to cause the leadscrew drivetrain 604 to rotate. The rotation of the leadscrew drivetrain 604 also causes the belt 616 to move and thereby activate the motor 610 operatively coupled thereto by rotating the motor 610. The activation of the motor 610 causes the energy storage mechanism 614 to be charged, through the rectifier 612. The energy storage mechanism 614 includes capacitors in this illustrated embodiment.

The circuit board 608 does not rotate or otherwise move in response to the rotation of the leadscrew drivetrain 604. The circuit board 608 in this illustrated embodiment has an opening 624 formed therein through which the leadscrew drivetrain 604 extends. The leadscrew drivetrain 604 is configured to rotate within the opening 624 without causing rotation of the circuit board 608.

The rotation of the leadscrew drivetrain 604 causes the magnet 606 to move with the leadscrew drivetrain 604 either proximally or distally. The distance between the magnet 606 and the Hall effect sensor 616, which is on the non-rotating, non-translating circuit board 608, therefore changes. The Hall effect sensor 616 will therefore sense a change. The IC circuit 620 is configured to receive an output from the Hall effect sensor 616 that indicates the change, thereby indicating a position of the leadscrew drivetrain 604. The IC circuit 620 is operatively coupled to the resonant antenna circuit 622 and is configured to cause data indicative of the position of the leadscrew drivetrain 604 to be communicated, via the resonant antenna circuit 622, to the robotic surgical system. The power stored in the energy storage mechanism 614 is configured to power the sensing circuit. A position of the leadscrew drivetrain 604 can therefore be communicated to the robotic surgical system without the surgical instrument receiving electrical power from the robotic surgical system to power the communication. A position of the leadscrew drivetrain 604 is indicative of a position of a firing sled at the end effector configured to push staples out of the end effector. The IC circuit 620 can be configured to calculate the position of the firing sled and communicate, via the resonant antenna circuit 622, the firing sled's position to the robotic surgical system.

In an exemplary embodiment, the tool driver is configured to provide a series of inputs to the firing stack 602 that alternately cause the leadscrew drivetrain 604 to rotate clockwise and counterclockwise in a dithering motion, thereby causing the belt 616 to move back and forth in alternate directions. The small oscillation of the dithering motion is sufficient to cause the belt 616 to move such that electrical energy is generated and stored in the energy storage mechanism 614 without the movement of the leadscrew drivetrain 604 being sufficient to cause any firing. The generator can therefore generate energy without a function of the surgical instrument being effectuated. The tool driver can be configured to begin the series of inputs to the tool housing 600 in response to the robotic surgical system sensing that the tool housing 600 has been releasably and replaceably coupled to the tool driver, which is a functionality (sensing tool housing coupling) the robotic surgical systems often have for use with surgical instruments. The tool driver can be configured to stop the series of inputs to the tool housing 600 in response to the energy storage mechanism 614 being fully charged. The IC circuit 620 can be configured to determine whether the energy storage mechanism 614 is fully charged and to communicate, via the resonant antenna circuit 622, data to the robotic surgical system indicating that the energy storage mechanism 614 is fully charged.

In some embodiments, instead of the robotic surgical system sensing that the tool housing 600 has been releasably and replaceably coupled to the tool driver as a trigger to begin providing a series of inputs to the firing stack 602, the robotic surgical system can be configured to move to a neutral state (or to remain in the neutral state) in which the inputs for dithering motion are provided to the tool housing 600 for charging purposes. The robotic surgical system can be configured to move from the neutral state to a firing state in which input(s) are provided to the tool housing 600 to cause firing.

Whether or not dithering motion of the leadscrew drivetrain 604 is used to charge the energy storage mechanism 614, input to the firing stack 602 to cause firing will cause the leadscrew drivetrain 604 to move and will thus cause charging of the energy storage mechanism 614. Generation of electrical power can therefore occur in this illustrated embodiment both without causing an output function of the surgical instrument and with causing the output function of the surgical instrument.

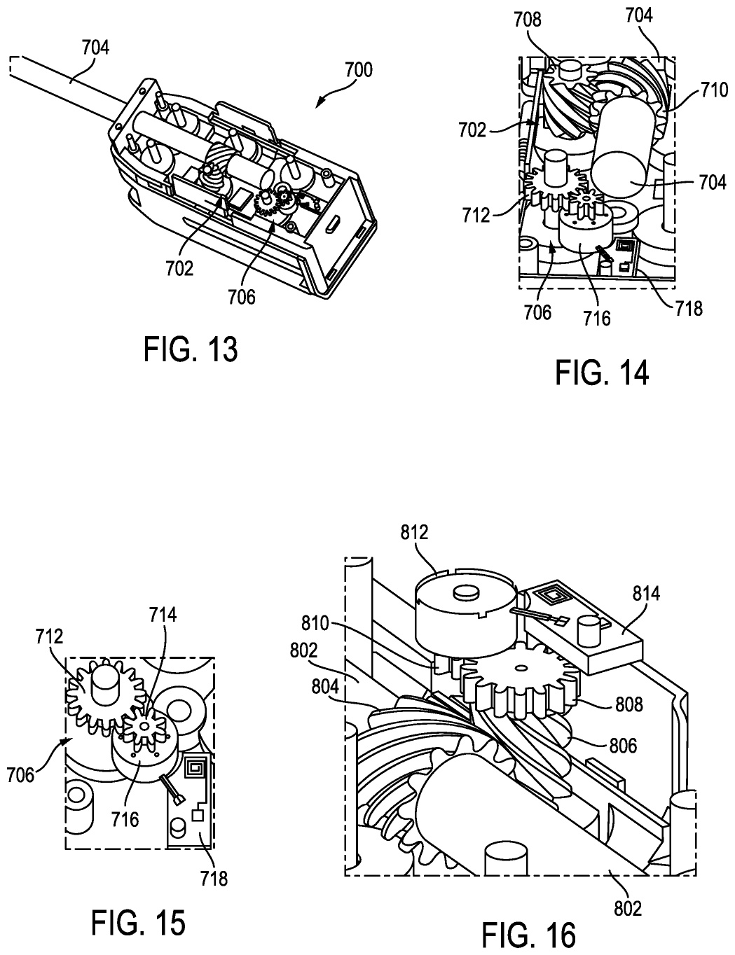

FIGS. 13-15 illustrate another embodiment of a tool housing 700, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing 700 is only partially shown in FIGS. 13-15. The tool housing 700 includes a generator configured to cause electrical power to be generated at the surgical instrument and that is contained in the tool housing 700. A tool driver to which the tool housing 700 is coupled is configured to cause the generator to generate and store power without causing an output function of the surgical instrument.

The tool housing 700 is configured to operatively couple to one or more motors of the tool driver via one or more input stacks of the tool housing, similar to that discussed above. In this illustrated embodiment, a rotation input stack 702 is configured to receive an input from the tool driver to cause rotation of the surgical instrument's elongate shaft 704 and end effector at a distal end of the elongate shaft 704. An energy generation input stack 706 is configured to receive an input from the tool driver to cause the generator to generate electrical energy on board the surgical instrument. Thus, the rotation input stack 702 is dedicated to a function of rotation and the energy generation input stack 706 is dedicated to a function of energy generation. Energy generation may therefore occur at the same time as shaft 704 and end effector rotation by inputs being provided to each of the rotation input stack 702 and the energy generation input stack 706, energy generation may occur without the shaft 704 and end effector rotating by input being provided to the energy generation input stack 706 but not to the rotation input stack 702, or shaft 704 and end effector rotation may occur without energy generation occurring by input being provided to the rotation input stack 702 but not to the energy generation input stack 706. The tool housing 700 is configured to receive four additional inputs at four additional input stacks that can be dedicated to a clinical function similar to the rotation input stack 702.

The rotation input stack 702 includes a first helical gear 708 configured to rotate in response to a mechanical, rotational input from the tool driver. The first helical gear 708 is operatively engaged with a second helical gear 710 that is operatively engaged with the shaft 704. Rotation of the first helical gear 708 causes the second helical gear 710 to rotate, which causes the shaft 704 to rotate about its longitudinal axis relative to the tool housing 700.

The energy generation input stack 706 includes a first gear 712 that is operatively coupled to a second gear 714 and that is configured to rotate in response to a mechanical, rotational input from the tool driver at the energy generation input stack 706. The first and second gear 712, 714 form a gear train. The second gear 714 is operatively coupled to a DC motor (such as a rotary permanent magnet DC motor) 716. Rotation of the first gear 712 causes the second gear 714 to rotate, which causes the DC motor 716 to rotate. The generator also includes a circuit board 718 to which the DC motor 716 is operatively coupled and that includes a rectifier and an energy storage mechanism. The rotation of the DC motor 716 causes energy to be stored at an energy storage mechanism, via the rectifier, to power a load, as discussed herein.

FIG. 16 illustrates another embodiment of a tool housing 800, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing 800 is only partially shown in FIG. 16. The tool housing 800 includes a generator configured to cause electrical power to be generated at the surgical instrument and that is contained in the tool housing 800. A tool driver to which the tool housing 800 is coupled is configured to cause the generator to generate power and cause an output function of the surgical instrument.

The tool housing 800 is configured to operatively couple to one or more motors of the tool driver via one or more input stacks of the tool housing, similar to that discussed above. In this illustrated embodiment, a rotation input stack is configured to receive an input from the tool driver to cause rotation of the surgical instrument's elongate shaft 802 and end effector at a distal end of the elongate shaft 802. The rotation input stack includes a first helical gear 804 configured to rotate in response to a mechanical, rotational input from the tool driver and operatively engaged with a second helical gear 806 that is operatively engaged with the shaft 802. Rotation of the first helical gear 804 causes the second helical gear 806 to rotate, which causes the shaft 802 to rotate about its longitudinal axis relative to the tool housing 800. The mechanical input to the rotation input stack is also configured to cause the generator to generate electrical power. The rotation input stack also includes a first gear 808 that is operatively coupled to a second gear 810 and that is configured to rotate in response to the mechanical input from the tool driver at the rotation input stack that also rotates the first helical gear 804. The first and second gear 808, 810 form a gear train. The second gear 810 is operatively coupled to a DC motor (such as a rotary permanent magnet DC motor) 812. Rotation of the first gear 808 causes the second gear 810 to rotate, which causes the DC motor 812 to rotate. The generator also includes a circuit board 814 to which the DC motor 812 is operatively coupled and that includes a rectifier and an energy storage mechanism. The rotation of the DC motor 812 causes energy to be stored at an energy storage mechanism, via the rectifier, to power a load, as discussed herein.

FIG. 17 illustrates another embodiment of a tool housing, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing is only partially shown in FIG. 17. The tool housing of FIG. 17 includes a generator configured to cause electrical power to be generated at the surgical instrument and that is contained in the tool housing. A tool driver to which the tool housing of FIG. 17 is coupled is configured to cause the generator to generate power and cause an output function of the surgical instrument.

The tool housing of FIG. 17 is configured to operatively couple to one or more motors of the tool driver via one or more input stacks of the tool housing, similar to that discussed above. In this illustrated embodiment, an input stack 900 includes a coupling element 902 configured to receive an input from the tool driver. The input is configured to cause a function of the surgical instrument, as discussed herein. The coupling element 902 in this illustrated embodiment includes a gear with teeth configured to operatively engage corresponding teeth of the motor. The input stack 900 also includes a drum (also referred to herein as a “capstone”) 904. The drum 904 is configured to rotate about a longitudinal axis 904a of the drum 904 that also defines a longitudinal axis of the input stack 900. A wire or cable 906 is coiled around the drum 904 with ends of the wire or cable 906 extending distally from the drum 904. Rotation of the capstone 904 is configured to cause longitudinal movement of the wire or cable 906 (proximal or distal movement as shown by arrows 910 depending on a direction of the drum's rotation with one end of the wire or cable 906 translating in one direction and the other end of the wire or cable 906 translating in the opposite direction) and thus effect a function of the surgical instrument, such as opening of, closing of, or articulating the end effector.

In this illustrated embodiment, the input stack 900 also includes a DC motor 908 (such as a rotary permanent magnet DC motor) of the generator. The input to the input stack 900 from the tool driver that causes the input stack 900 to rotate thus causes the motor 908 to rotate. The rotation of the motor 908 causes energy to be generated and stored as discussed herein, for example as discussed with respect to the generator including the motor 102 of FIG. 6, the generator including the motor 202 of FIG. 7, or the generator including the motor 302 of FIG. 8. The motor 908 is operatively coupled to a load circuit 912 configured to be powered by the generated electrical energy, as also discussed herein.

FIG. 18 illustrates another embodiment of a tool housing 1000, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing of FIG. 18 includes a generator configured to cause electrical power to be generated at the surgical instrument 1002. A tool driver to which the tool housing of FIG. 18 is coupled is configured to cause the generator to generate power without causing an output function of the surgical instrument 1002. The embodiment of FIG. 18 is similar to the embodiment of FIG. 12 in that each embodiment can use backlash to generate electrical energy at the tool housing. In this illustrated embodiment, the backlash is linear backlash.

The surgical instrument 1002 includes an elongate shaft 1004 and an end effector 1006. The end effector 1006 in this illustrated embodiment includes a pair of opposed jaws. FIG. 18 illustrates the surgical instrument 1002 releasably coupled to a robotic arm 1008, e.g., the robotic arm 52 of FIG. 3, of a robotic surgical system. The robotic arm 1008 includes an entry guide 1010, which is a trocar in this illustrated embodiment, and which can be a part of or can be removably coupled to the robotic arm 1008. The elongate shaft 1004 and the end effector 1006 are shown within the entry guide 1010 in FIG. 18.

The robotic arm 1008 also includes a carriage 1012. The carriage 1012 is configured to slide longitudinally back and forth (proximally and distally as shown by an arrow 1014) to facilitate generation of electrical power, as discussed further below. The carriage 1012 is shown as a rectangular block in this illustrated embodiment but can have other configurations.

The tool housing 1000 houses therein a spring 1016, a metallic (e.g., copper) coil 1018, a permanent magnet 1020, a plunger 1022, and a load 1024. The spring 1016 is a coil spring in this illustrated embodiment but can have another configuration. The magnet 1020 is a single magnet in this illustrated embodiment but can be a plurality of magnets. The magnet 1020 is attached to the plunger 1022 in a fixed position relative thereto. The coil 1018 is coiled around the plunger 1022 within the tool housing 1000 such that the plunger 1022 can move longitudinally proximally and distally within an interior of the coil 1018 relative to the coil 1018.

The carriage 1012 is configured to move between a resting position and a generating position to cause the generator to generate electrical power. In the resting position, which is shown in FIG. 18, a proximal portion of the plunger 1022 extends proximally out of the tool housing 1000, the plunger 1022 is not in contact with the robotic arm 1008, the spring 1014 is uncompressed, and a proximal surface of the carriage 1012 is in contact with a distal surface of the tool housing 1000. In other embodiments, in the resting position, the proximal surface of the carriage 1012 can be distal to the distal surface of the tool housing 1000 and not be in contact with the distal surface of the tool housing 1000. In the generating position, the plunger 1022 is in contact with the robotic arm 1008, the spring 1014 is compressed, and the proximal surface of the carriage 1012 is in contact with the distal surface of the tool housing 1000. The carriage 1012 is in a more proximal positon in the generating position than in the resting position.

The carriage 1012 moving in a proximal direction from the resting position to the generating position causes the carriage 1012 to push the tool housing 1000 proximally due to the contact of the proximal surface of the carriage 1012 with the distal surface of the tool housing 1000. The plunger 1022 moves proximally with the tool housing 1000 until a proximal end of the plunger 1022 abuts a distal surface 1026 of the robotic arm 1008. The tool housing 1000 continues to move proximally while the spring 1014 compresses with the plunger 1022 abutting the distal surface 1026 of the robotic arm 1008. The tool housing 1000 is thus moving proximally relative to the plunger 1022 and therefore also relative to the magnet 1020. The tool housing 1000, including the coil 1018 contained therein, moving relative to the magnet 1020 causes the magnet 1020 to interact with the copper coil 1018 and generate an electromagnetic field, which generates electrical power for the load 1024, as discussed herein. The carriage 1012 reaches the generating position when a proximal surface of the tool housing 1000 abuts the distal surface 1026 of the robotic arm 1008, which effectively prevents the tool housing 1000 from moving further proximally. The carriage 1012 moving in a distal direction from the generating position to the resting position similarly causes the magnet 1020 to interact with the copper coil 1018.

The robotic surgical system is configured to control the movement of the carriage 1012 between the resting and generating positions. In an exemplary embodiment, the robotic surgical system is configured to control movement of the carriage 1012 such that the carriage 1012 moves repeatedly back and forth between the resting and generating positions in a dithering motion. The generator can therefore generate energy without a function of the surgical instrument being effectuated.

In an exemplary embodiment, the robotic surgical system is configured to control movement of the carriage 1012 between the resting and generating positions when the end effector 1006 of the surgical instrument 1002 is located within the entry guide 1010. The end effector 1006 being located within the entry guide 1010 indicates that the surgical instrument 1002 is not in use on tissue of a patient or on other matter at a surgical site. The surgical instrument 1002 can thus be oscillated back and forth as the carriage 1012 moves back and forth between the resting and generating positions without affecting use of the surgical instrument 1002 during performance of a surgical procedure on the patient. The robotic surgical system can be configured to control movement of the carriage 1012 between the resting and generating positions when the surgical instrument 1002 is not in use with a patient even if the end effector 1006 is not located within the entry guide 1010 and is located distal to the entry guide 1010, if the end effector 1006 is clear of tissue and other matter at the surgical site that could interfere with the backlash motion.

The carriage 1012 is configured to move between the resting position and a non-generating position. The carriage 1012 in the non-generating position corresponds to any location of the carriage 1012 distal to the resting position. The surgical instrument 1002 is more distally advanced through the entry guide 1010 with the carriage 1012 in the non-generating position. The carriage 1012 is configured to passively move distally from the resting position to the non-generating position by the tool housing 1000 pushing distally against the carriage 1012 as the surgical instrument 1002 is moved distally through the entry guide 1010. The robotic surgical system is configured to cause the carriage 1012 to move from the non-generating position to the resting position to ready the carriage 1012 for assisting in energy generation as discussed above.

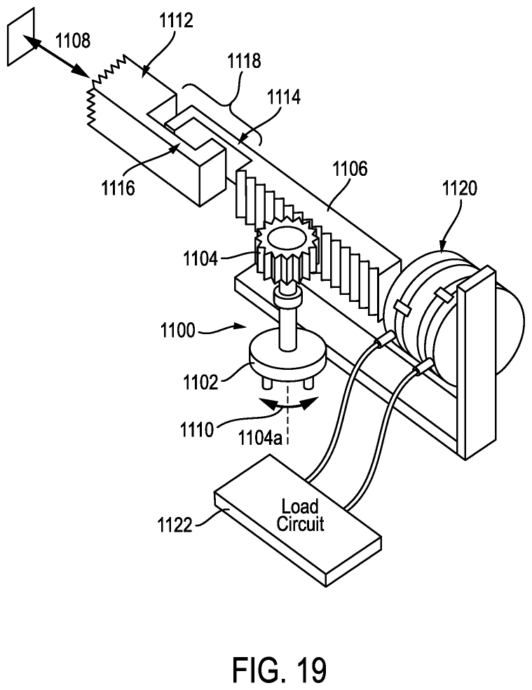

FIG. 19 illustrates another embodiment of a tool housing, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing is only partially shown in FIG. 19. The tool housing of FIG. 19 includes a generator configured to cause electrical power to be generated at the surgical instrument. A tool driver to which the tool housing of FIG. 19 is coupled is configured to cause the generator to generate power without causing an output function of the surgical instrument. The embodiment of FIG. 19 is similar to the embodiments of FIGS. 12 and 18 in that each embodiment can use backlash to generate electrical energy at the tool housing. In this illustrated embodiment, the backlash is linear backlash.

The tool housing of FIG. 19 is configured to operatively couple to one or more motors of the tool driver via one or more input stacks of the tool housing, similar to that discussed above. In this illustrated embodiment, an input stack 1100 includes a coupling element 1102 configured to receive an input from the tool driver. The input is configured to cause a function of the surgical instrument, as discussed above. The coupling element 1102 in this illustrated embodiment includes a gear with teeth configured to operatively engage corresponding teeth of the motor. The input stack 1100 also includes a pinion 1104. The pinion 1104 is configured to rotate about a longitudinal axis 1104a of the pinion 1104 that also defines a longitudinal axis of the input stack 1100.

The pinion 1104 is operatively engaged with teeth of a rack 1106. Rotation of the pinion 1104, e.g., in response to a mechanical, rotational input from the tool driver to the input stack 1000, is configured to cause longitudinal movement of the rack 1106 (proximal or distal movement shown by an arrow 1108 depending on a direction of the pinion's rotation shown by an arrow 1110) and thus effect a function of the surgical instrument, such as opening or closing of an end effector, translating a cutting element, or firing staples, by longitudinally moving an actuation shaft 1112. The teeth that engage the pinion 1104 are in a proximal portion of the rack 1106. A distal portion of the rack 1106 lacks teeth and defines a first hook 1114. A proximal portion of the actuation shaft 1112 defines a second hook 1116 that faces the first hook 1114. The first and second hooks 1114, 1116 define a backlash area 1118 in which the rack 1106 is configured to move relative to the actuation shaft 1112 without causing the actuation shaft 1112 to move longitudinally (proximally or distally) and thus for the rack 1106 to move without effecting a function of the surgical instrument

The rack 1106 is configured to move between a resting position and a generating position to cause the generator to generate electrical power. The rack 1106 is in a more proximal positon in the generating position than in the resting position. The generator includes a piezoelectric stack 1120 that is located distal to the rack 1106. In the resting position, which is shown in FIG. 19, the rack 1106 is not in contact with the piezoelectric stack 1120 (e.g., is located proximal to the piezoelectric stack 1120), a proximal surface of the first hook 1114 is in contact with a distal surface of the second hook 1116 at a front or proximal end of the backlash area 1118, and a distal surface of the first hook 1114 is not in contact with a proximal surface of the second hook 1116. In other embodiments, in the resting position, the proximal surface of the rack 1106 can be distal to the distal surface of the actuation shaft 1112 and not be in contact with the distal surface of the actuation shaft 1112. In the generating position, the rack 1106 (e.g., a distal surface of the rack 1106) is in contact with the piezoelectric stack 1120, the proximal surface of the first hook 1114 is not in contact with the distal surface of the second hook 1116, and the distal surface of the first hook 1114 is in contact with the proximal surface of the second hook 1116 at a rear or distal end of the backlash area 1118. The rack 1106 colliding with the piezoelectric stack 1120 when the rack 1106 reaches the generating position induces an electric potential at the piezoelectric stack 1120, which generates electrical power for a load circuit 1122, as discussed herein.

The robotic surgical system is configured to control the movement of the rack 1106 between the resting and generating positions with inputs to the input stack 1100. In an exemplary embodiment, the robotic surgical system is configured to control movement of the rack 1106 such that the rack 1106 moves repeatedly back and forth between the resting and generating positions in a dithering motion, e.g., by providing inputs the alternately cause the input stack 1100 to rotate clockwise and counterclockwise. The generator can therefore generate energy without a function of the surgical instrument being effectuated because the rack 1106 is moving within the backlash area 1118 such that the actuation shaft 1112 is not moved longitudinally even though the rack 1106 is moving longitudinally.

In an exemplary embodiment, the robotic surgical system is configured to control movement of the rack 1106 between the resting and generating positions when the end effector of the surgical instrument is located within an entry guide, similar to that discussed above regarding the embodiment of FIG. 18.

The rack 1106 is configured to move between the resting position and a non-generating position. The surgical instrument is thus more distally advanced through the entry guide with the rack 1106 in the non-generating position. The non-generating position of the rack 1106 corresponds to a function of the surgical instrument being effectuated because the rack 1106 has moved distally enough to push the actuation shaft 1112 distally.

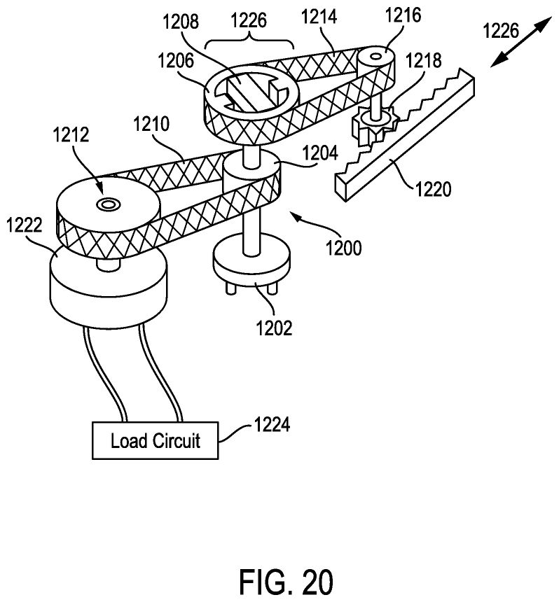

FIG. 20 illustrates another embodiment of a tool housing, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing is only partially shown in FIG. 20. The tool housing of FIG. 20 includes a generator configured to cause electrical power to be generated at the surgical instrument. A tool driver to which the tool housing of FIG. 20 is coupled is configured to cause the generator to generate power without causing an output function of the surgical instrument. The embodiment of FIG. 20 is similar to the embodiments of FIGS. 12, 18, and 19 in that each embodiment can use backlash to generate electrical energy at the tool housing. In this illustrated embodiment, the backlash is rotational backlash.

The tool housing of FIG. 20 is configured to operatively couple to one or more motors of the tool driver via one or more input stacks of the tool housing, similar to that discussed above. In this illustrated embodiment, an input stack 1200 includes a coupling element 1202 configured to receive an input from the tool driver. The input is configured to cause a function of the surgical instrument, as discussed herein. The coupling element 1202 in this illustrated embodiment includes a gear with teeth configured to operatively engage corresponding teeth of the motor. The input stack 1200 also includes a first belt gear 1204, a second belt gear 1206, and a paddle 1208. The first belt gear 1204 is operatively engaged with a first belt 1210 that is also operatively engaged with a third belt gear 1212. The second belt gear 1206 is operatively engaged with a second belt 1214 that is also operatively engaged with a fourth belt gear 1216. The fourth belt gear 1216 is operatively engaged with a pinion 1218 that is operatively engaged with teeth of a rack 1220.

In response to an input from the tool driver to the input stack 1200, the input stack 1200 including the coupling element 1202, the first belt gear 1204, and the paddle 1208 are configured to rotate. The rotation of the first belt gear 1204 causes movement of the first belt 1210, which causes the third belt gear 1212 to rotate. The third belt gear 1212 is operatively coupled to a DC motor 1222 (such as a rotary permanent magnet DC motor) of the generator such that the rotation of the third belt gear 1212 causes the motor 1222 to rotate. The rotation of the motor 1222 causes energy to be generated and stored as discussed herein, for example as discussed with respect to the generator including the motor 102 of FIG. 6, the generator including the motor 202 of FIG. 7, or the generator including the motor 302 of FIG. 8. The motor 1222 is operatively coupled to a load circuit 1224 configured to be powered by the generated electrical energy, as also discussed herein.

The second belt gear 1206 only sometimes rotates in response to input to the input stack 1200. The second belt 1214, the fourth belt gear 1216, the pinion 1218, and the rack 1220 therefore only sometimes move in response to input to the input stack 1200. The second belt gear 1206 and the paddle 1208 define a backlash area 1226 in which the paddle 1208 is configured to rotate relative to the second belt gear 1206 without causing the second belt gear 1206 to rotate and thus without any of the second belt 1214, the fourth belt gear 1216, the pinion 1218, and the rack 1220 moving and without effecting a function of the surgical instrument. The paddle 1208 moving only in the backlash area 1226 (e.g., not moving beyond the backlash area 1226) corresponds to the generator generating energy without effecting a function of the surgical instrument. The paddle 1208 moving beyond the backlash area 1226 corresponds to the generator generating energy with a function of the surgical instrument being effected.

The robotic surgical system is configured to control the energy generation with the paddle 1208 moving only in the backlash area 1226. In an exemplary embodiment, the robotic surgical system is configured to control movement of the paddle 1208 such that the paddle 1208 rotates repeatedly clockwise and counterclockwise in the backlash area 1226 in a dithering motion, e.g., by providing inputs the alternately cause the input stack 1200 to rotate clockwise and counterclockwise. The generator can therefore generate energy without a function of the surgical instrument being effectuated because the paddle 1208 is rotating within the backlash area 1226 such that the second belt gear 1206 does not rotate to transfer movement to the rack 1220.

In an exemplary embodiment, the robotic surgical system is configured to control movement of the paddle 1208 within the backlash area 1226 when the end effector of the surgical instrument is located within an entry guide, similar to that discussed above regarding the embodiment of FIG. 18.

The paddle 1208 rotating beyond the backlash area 1222 causes the paddle 1208 to engage the second belt gear 1206 so as to push the second belt gear 1206 in rotation corresponding to the paddle's rotation. The rotation of the second belt gear 1206 causes movement of the second belt 1214, which causes the fourth belt gear 1216 to rotate. The rotation of the fourth belt gear 1216 causes the pinion 1218 to rotate. The rotation of the pinion 1218 causes the rack 1220 to move longitudinally either proximally or distally, as shown by an arrow 1226, depending on a direction of the input stack's rotation. With the paddle 1208 rotating beyond the backlash area 1222, the first belt gear 1204 is also rotating such that energy generator can occur when a function of the surgical instrument is being effected.

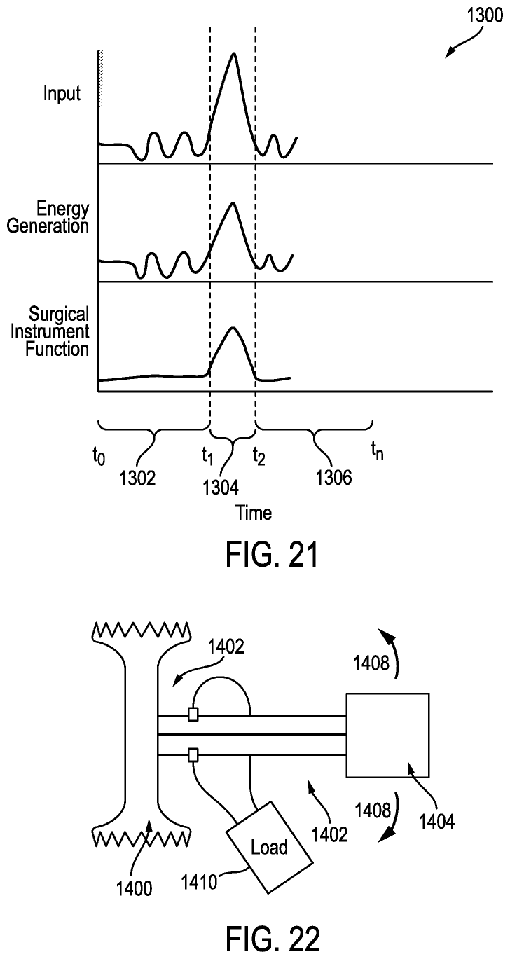

FIG. 21 shows one possible graphical representation 1300 plotting each of input, energy generation, and surgical instrument function versus time for embodiments configured to use backlash such as the tool housing 600 of FIG. 12, the tool housing 1000 of FIG. 18, the tool housing of FIG. 19, and the tool housing of FIG. 20. In a first time period 1302 from time t₀ to time t₁, a tool driver is providing input to a tool housing, e.g., to an input stack thereof, such that energy generation occurs. The input is shown as oscillating in the first time period 1302, reflecting the back and forth motion of backlash. In a second time period 1304 from time t₁ to time t₂, the tool driver is providing input to the tool housing such that energy generation occurs and a function of the surgical instrument is effected. In a third time period 1306 starting at time t₂, the tool driver is providing input to the tool housing such that energy generation occurs. The input is shown as oscillating in the third time period 1306, reflecting the back and forth motion of backlash. A function of the surgical instrument is not effected in the third time period 1306. The third time period 1306 in which energy generation occurs without a function of the surgical instrument being effected can continue until time tₙ, which is when use of the surgical instrument ends in the surgical procedure. Alternatively, periods of energy generation and surgical instrument function similar to the second time period 1304 can alternate any number of times with periods of energy generation without surgical instrument function similar to the first and third times periods 1302, 1306 until time tₙ.

In some embodiments, an input of a robotic surgical system to a tool housing of a surgical instrument can be configured to cause a generator contained in the tool housing to generate energy in response to any input from the robotic surgical system that causes the tool housing to move. The generator in such embodiments need not be operatively coupled to any input stack of the surgical instrument. Instead, the generator can be attached to an internal surface of the tool housing and be configured to be activated in response to whichever input stack(s) cause movement of the tool housing in response to a tool driver's input thereto. One example of such an input is an input to cause longitudinal translation of the surgical instrument's elongate shaft and end effector since the tool housing longitudinally translates with the elongate shaft and end effector. Additionally, in such embodiments, the generator is configured to generate energy without being coupled to a robotic surgical system. Natural movement of the tool housing, such as during transport of the tool housing, while a user holds and moves the surgical instrument toward being coupled to a robotic surgical system, etc., is configured to cause the generator to generate energy in response to the movement of the tool housing. The surgical instrument may therefore have energy stored onboard ready for use before being coupled to a robotic surgical system.

FIG. 22 illustrates another embodiment of a tool housing 1400, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing 1400 is only partially shown in FIG. 22. The tool housing 1400 of FIG. 22 includes a generator configured to cause electrical power to be generated at the surgical instrument. A tool driver to which the tool housing 1400 of FIG. 22 is coupled is configured to cause the generator to generate power with or without causing an output function of the surgical instrument, depending on what causes the tool housing 1400 to move. In this illustrated embodiment, the generator is attached to an internal surface 1402 of the tool housing 1400 and is configured to generate energy in response to any input that causes the tool housing to move. A bimetallic strip 1404 is attached to the tool housing's internal surface 1402 at one end of the bimetallic strip 1404 and is attached to a free mass 1406 at the other, opposite end of the bimetallic strip 1404. The internal surface 1402 can be anywhere within the tool housing 1400 wherever there is sufficient space within the tool housing 1400. In response to movement of the tool housing 1400, whether by natural movement or in response to an input from a robotic surgical system, the mass 1406 will move, as shown by arrows 1408. The movement of the mass 1406 causes deflection of the bimetallic strip 1404, e.g., in response to reaction force of the mass 1406, similar to a spring's movement. The deflection of the bimetallic strip 1404 causes an electric potential. The bimetallic strip 1404 is operatively coupled to a load 1410 configured to be powered by the generated electrical energy, as discussed herein.

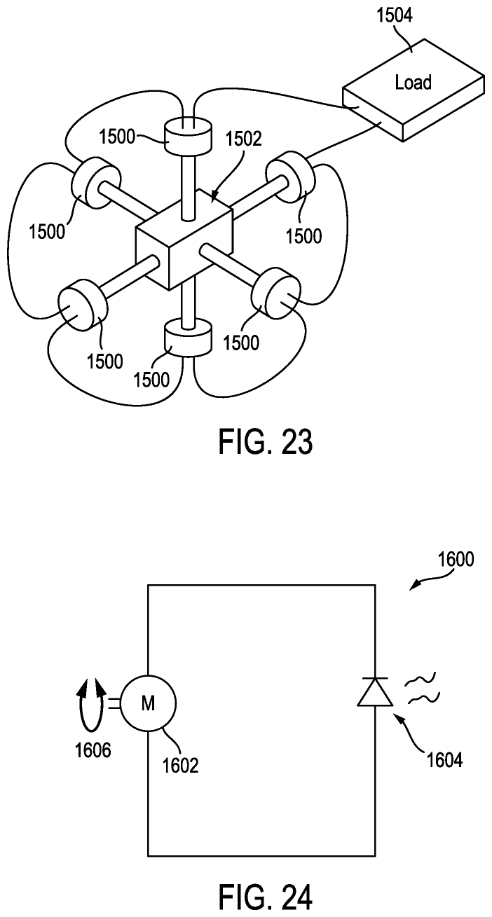

FIG. 23 illustrates another embodiment of a tool housing, e.g., the tool housing 16 of FIG. 1 or the tool housing 68 of FIGS. 3 and 4, configured to be releasably and replaceably coupled to a tool driver, e.g., the tool driver 18 of FIG. 1 or the tool driver 56 of FIGS. 3 and 5. The tool housing is only partially shown in FIG. 23. The tool housing of FIG. 23 includes a generator configured to cause electrical power to be generated at the surgical instrument. A tool driver to which the tool housing of FIG. 23 is coupled is configured to cause the generator to generate power with or without causing an output function of the surgical instrument, depending on what causes the tool housing to move. In this illustrated embodiment, the generator is attached to an internal surface of the tool housing and is configured to generate energy in response to any input that causes the tool housing to move. An array of piezoelectric stacks 1500 is attached to the tool housing's internal surface. The internal surface of the tool housing can be anywhere within the tool housing wherever there is sufficient space within the tool housing. Each of the piezoelectric stacks 1500 is also attached to a free mass 1502. In response to movement of the tool housing, whether by natural movement or in response to an input from a robotic surgical system, the mass 1502 will move. The movement of the mass 1502 causes pressure on various ones of the piezoelectric stacks 1500, which induces an electric potential, which generates electrical power for a load 1504, as discussed herein.

In some instances, a power consumption of a surgical instrument's load may not exceed mechanical input being provided to the surgical instrument from a motor of a robotic surgical system, e.g., being provided from a motor of the tool driver 18 to the tool housing 16 of FIG. 1 or from one of the motors 64 of the tool driver 56 to the tool housing 68 of FIGS. 3-5. In such instances, the surgical instrument does not need to store electrical power generated in response to the mechanical input. The electrical power can simply be used to power the load without storing the electrical power.

FIG. 24 illustrates one embodiment of a circuit 1600 configured to generate electrical power without storing the power. The circuit 1600 includes a DC motor 1602 and a light 1604. The DC motor 1602 is configured to be operably coupled to a mechanical source. The mechanical source includes a component of an input stack of a surgical instrument's tool housing that is configured to rotate in response to an input thereto from a tool driver. The DC motor 1602 is configured to correspondingly rotate in response to the rotation of the mechanical source, such as by being directly attached to the mechanical source or by being indirectly coupled to the mechanical source using a belt operably coupled to the mechanical source and the DC motor 1602 similar to the belts discussed above. The light 1604 is a load configured to be illuminated for a first polarity, e.g., the DC motor 1602 rotating in a first direction 1606 in response to an input in the first direction 1606, and to not be illuminated for a second, opposite polarity, e.g., the DC motor 1602 rotating a second, opposite direction. The first direction 1606 is counterclockwise in this illustrated embodiment but could instead be clockwise.

The first and second directions of rotation are indicative of the function being caused by the input to the tool housing that is causing the rotation of the mechanical source and thus the rotation of the motor 1602. The light being illuminated or not thus indicates the function being performed. For example, rotation in the first direction can indicate proximal advancement of a cutting element along the surgical instrument's end effector such that the light 1604 being illuminated indicates that cutting of tissue held by the end effector is occurring, and rotation in the second direction can indicate distal retraction of the cutting element along the surgical instrument's end effector such that the light 1604 not being illuminated indicates that cutting of tissue held by the end effector is not occurring. For another example, rotation in the first direction can indicate proximal advancement of a firing sled along the surgical instrument's end effector such that the light 1604 being illuminated indicates that stapling of tissue held by the end effector is occurring, and rotation in the second direction can indicate distal retraction of the firing sled along the surgical instrument's end effector such that the light 1604 not being illuminated indicates that stapling of tissue held by the end effector is not occurring.

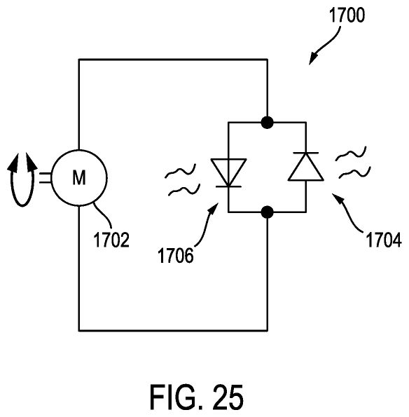

FIG. 25 illustrates another embodiment of a circuit 1700 configured to generate electrical power without storing the power. The circuit 1700 includes a DC motor 1702, a first light 1704, and a second light 1706. The circuit 1700 of FIG. 25 is configured and used similar to the circuit 1600 of FIG. 24 except that the circuit 1700 includes two lights 1704, 1706 instead of one light 1604. The first light 1704 is configured to be illuminated for a first polarity, e.g., the DC motor 1702 rotating in a first direction, and to not be illuminated for a second, opposite polarity, e.g., the DC motor 1702 rotating a second, opposite direction. The second light 1706 is configured to be illuminated for the second polarity and to not be illuminated for the first polarity. As discussed above, the first and second directions of rotation are indicative of the function being caused by the input to the tool housing that is causing the rotation of the mechanical energy source and thus the rotation of the motor 1702. The first and second lights 1704, 1706 being illuminated or not thus indicates the function being performed. For example, rotation in the first direction can indicate proximal advancement of a cutting element along the surgical instrument's end effector such that the first light 1704 being illuminated indicates that cutting of tissue held by the end effector is occurring, and rotation in the second direction can indicate distal retraction of the cutting element along the surgical instrument's end effector such that the second light 1706 being illuminated indicates that cutting of tissue held by the end effector is not occurring. For another example, rotation in the first direction can indicate proximal advancement of a firing sled along the surgical instrument's end effector such that the first light 1704 being illuminated indicates that stapling of tissue held by the end effector is occurring, and rotation in the second direction can indicate distal retraction of the firing sled along the surgical instrument's end effector such that the second light 1706 being illuminated indicates that stapling of tissue held by the end effector is not occurring.

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

What is claimed is:

1. A surgical system, comprising:
a tool housing of a surgical instrument configured to releasably couple to a tool driver of a robotic surgical system, the tool housing being configured to receive a mechanical, rotational input from the tool driver with the tool housing releasably coupled to the tool driver; and
a generator contained in the tool housing;
wherein the receipt of the mechanical, rotational input is configured to cause the generator to generate electrical energy configured to be used onboard the surgical instrument.

2. The system of claim 1, wherein the generator includes a motor configured to rotate to generate the electrical energy and includes an energy storage mechanism configured to store the generated electrical energy prior to the use of the generated electrical energy onboard the surgical instrument.

3. The system of claim 2, further comprising a load contained in the tool housing and configured to be powered with the electrical energy stored in the energy storage mechanism.

4. The system of claim 1, wherein the generated electrical energy is configured to be used onboard the surgical instrument without storing the generated electrical energy onboard the surgical instrument.

5. The system of claim 1, wherein the surgical instrument is not configured to receive electrical energy from the robotic surgical system via a wired connection or a wireless connection.

6. The system of claim 1, wherein the input is from a motor of the tool driver, the input is configured to cause an input stack of the surgical instrument to rotate, and the rotation of the input stack is configured to drive the generator to generate the energy.

7. The system of claim 1, wherein the receipt of the mechanical, rotational input is configured to cause the generator to generate the electrical energy and to cause the surgical instrument to perform a clinical function.

8. The system of claim 1, wherein the receipt of the mechanical, rotational input is configured to cause the generator to generate the electrical energy without causing the surgical instrument to perform a clinical function.

9. A surgical system, comprising:
a tool housing of a surgical instrument configured to releasably couple to a tool driver of a robotic surgical system, the tool housing being configured to receive an input from the tool driver with the tool housing releasably coupled to the tool driver, the input being configured to cause the surgical instrument to perform a clinical function; and
a generator contained in the tool housing;
wherein the receipt of the input is configured to cause the generator to generate electrical energy that is configured to be used onboard the surgical instrument.

10. The system of claim 9, wherein the receipt of the input is configured to cause the generator to generate the electrical energy and to cause the surgical instrument to perform the clinical function.

11. The system of claim 9, wherein the receipt of the input is configured to cause the generator to generate the electrical energy without causing the surgical instrument to perform the clinical function.

12. The system of claim 11, wherein the input is configured to cause movement of a mechanical element within the tool housing;
the movement of the mechanical element being within a backlash area is configured to cause the generator to generate the electrical energy without causing the surgical instrument to perform the clinical function; and
the movement of the mechanical element being beyond the backlash area is configured to cause the generator to generate the electrical energy and to cause the surgical instrument to perform the clinical function.

13. The system of claim 9, wherein the input is a mechanical, rotational input.

14. The system of claim 9, wherein the generator includes a motor configured to rotate to generate the electrical energy and includes an energy storage mechanism configured to store the generated electrical energy prior to the use of the generated electrical energy onboard the surgical instrument.

15. The system of claim 14, further comprising a load contained in the tool housing and configured to be powered with the electrical energy stored in the energy storage mechanism.

16. The system of claim 9, wherein the generated electrical energy is configured to be used onboard the surgical instrument without storing the generated electrical energy onboard the surgical instrument.

17. The system of claim 9, wherein the input is from a motor of the tool driver, the input is configured to cause an input stack of the surgical instrument to rotate, and the rotation of the input stack is configured to drive the generator to generate the energy.

18. A surgical method, comprising:
receiving, at a tool housing of a surgical instrument releasably coupled to a tool driver of a robotic surgical system, a mechanical input from the tool driver;
wherein the receipt of the mechanical input causes a generator contained in the tool housing to generate electrical energy used onboard the surgical instrument.

19. The method of claim 18, wherein the generator includes a motor that rotates to generate the electrical energy and includes an energy storage mechanism that stores the generated electrical energy.

20. The method of claim 18, wherein the generated electrical energy is used onboard the surgical instrument without storing the generated electrical energy onboard the surgical instrument.

21. The method of claim 18, wherein the input is from a motor of the tool driver, the input causes an input stack of the surgical instrument to rotate, and the rotation of the input stack drives the generator to generate the energy.

22. The method of claim 18, wherein the receipt of the mechanical input causes the generator to generate the electrical energy and causes the surgical instrument to perform a clinical function.

23. The method of claim 18, wherein the receipt of the mechanical input causes the generator to generate the electrical energy without causing the surgical instrument to perform a clinical function.
